SEQUENCE LISTING (98 sequences)

SEQ ID NO: 1 - RNA, 22 nt, human
ugaccgauuucuccugguguuc

SEQ ID NO: 2 - RNA, 88 nt, human
aucucuuacacaggcugaccgauuucuccugguguucagagucuguuuuugucuagcaccauuugaaaucgguuaugauguaggggga

SEQ ID NO: 3 - RNA, 22 nt, human
aaaagcuggguugagagggcga

SEQ ID NO: 4 - RNA, 82 nt, human
gcuucgcuccccuccgccuucucuucccgguucuucccggagucgggaaaagcuggguugagagggcgaaaaaggaugaggu

SEQ ID NO: 5 - RNA, 22 nt, human
ugagguaguagguugugugguu

SEQ ID NO: 6 - RNA, 83 nt, human
cggggugagguaguagguugugugguuucagggcagugauguugccccucggaagauaacuauacaaccuacugccuucccug

SEQ ID NO: 7 - RNA, 28 nt, human
ucacaaugcugacacucaaacugcugac

SEQ ID NO: 8 - RNA, 104 nt, human
cugugcuuuguguguuggaggaugaaaguacggagugauccaucggcuaagugucuugucacaaugcugacacucaaacugcugacagcacacguuuuucacag

SEQ ID NO: 9 - RNA, 22 nt, human
uggcagugucuuagcugguugu

SEQ ID NO: 10 - RNA, 110 nt, human
ggccagcugugaguguuucuuuggcagugucuuagcugguuguugugagcaauaguaaggaagcaaucagcaaguauacugcccuagaagugcugcacguuguggggccc

SEQ ID NO: 11 - RNA, 21 nt, human
caacaccagucgaugggcugu

SEQ ID NO: 12 - RNA, 72 nt, human
ugucggguagcuuaucagacugauguugacuguugaaucucauggcaacaccagucgaugggcugucugaca

SEQ ID NO: 13 - RNA, 22 nt, human
aaaccguuaccauuacugaguu

SEQ ID NO: 14 - RNA, 72 nt, human
cuugggaauggcaaggaaaccguuaccauuacugaguuuaguaaugguaaugguucucuugcuauacccaga

SEQ ID NO: 15 - RNA, 22 nt, human
ucguaccgugaguaauaaugcg

SEQ ID NO: 16 - RNA, 85 nt, human
cgcuggcgacgggacauuauuacuuuugguacgcgcugugacacuucaaacucguaccgugaguaauaaugcgccguccacggca

SEQ ID NO: 17 - RNA, 22 nt, human
uccuguacugagcugccccgag

SEQ ID NO: 18 - RNA, 68 nt, human
gcauccuguacugagcugccccgaggcccuucaugcugcccagcucggggcagcucaguacaggauac

SEQ ID NO: 19 - RNA, 22 nt, human
ggcuggcucgcgaugucuguuu

SEQ ID NO: 20 - RNA, 97 nt, human
ccaguggcgcaauggauaacgcgucugacuacggaucagaagauucuagguucgacuccuggcuggcucgcgaugucuguuuugccacacuugaccc

SEQ ID NO: 21 - RNA, 22 nt, human
ugucaguuugucaaauacccca

SEQ ID NO: 22 - RNA, 110 nt, human
ccuggccuccugcagugccacgcuccguguauuugacaagcugaguuggacacuccaugugguagagugucaguuugucaaauaccccaagugcggcacaugcuuaccag

SEQ ID NO: 23 - RNA, 22 nt, human
uuugguccccuucaaccagcug

SEQ ID NO: 24 - RNA, 88 nt, human
acaaugcuuugcuagagcugguaaaauggaaccaaaucgccucuucaauggauuugguccccuucaaccagcuguagcuaugcauuga

SEQ ID NO: 25 - RNA, 102 nt, human
gggagccaaaugcuuugcuagagcugguaaaauggaaccaaaucgacuguccaauggauuugguccccuucaaccagcuguagcugugcauugauggcgccg

SEQ ID NO: 26 - RNA, 22 nt, human
ucuacagugcacgugucuccag

SEQ ID NO: 27 - RNA, 68 nt, human
guguauucuacagugcacgugucuccaguguggcucggaggcuggagacgcggcccuguuggaguaac

SEQ ID NO: 28 - RNA, 22 nt, human
uuugguccccuucaaccagcua

SEQ ID NO: 29 - RNA, 119 nt, human
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 30 - RNA, 21 nt, human
gcgacccauacuugguuucag

SEQ ID NO: 31 - RNA, 96 nt, human
agaugugcucuccuggcccaugaaaucaagcgugggugagaccuggugcagaacgggaaggcgacccauacuugguuucagaggcugugagaauaa

SEQ ID NO: 32 - RNA, 23 nt, human
caaagugcucauagugcagguag

SEQ ID NO: 33 - RNA, 69 nt, human
aguaccaaagugcucauagugcagguaguuuuggcaugacucuacuguaguaugggcacuuccaguacu

SEQ ID NO: 34 - RNA, 22 nt, human
uagcuuaucagacugauguuga

SEQ ID NO: 35 - RNA, 72 nt, human; identical to SEQ ID NO: 12

SEQ ID NO: 36 - RNA, 23 nt, human
guccaguuuucccaggaaucccu

SEQ ID NO: 37 - RNA, 88 nt, human
caccuuguccucacgguccaguuuucccaggaaucccuuagaugcuaagauggggauuccuggaaauacuguucuugaggucaugguu

SEQ ID NO: 38 - RNA, 22 nt, human
aacccguagauccgaucuugug

SEQ ID NO: 39 - RNA, 81 nt, human
cccauuggcauaaacccguagauccgaucuuguggugaaguggaccgcacaagcucgcuucuaugggucugugucagugug

SEQ ID NO: 40 - RNA, 22 nt, human
cugugcgugugacagcggcuga

SEQ ID NO: 41 - RNA, 110 nt, human
acccggcagugccuccaggcgcagggcagccccugcccaccgcacacugcgcugccccagacccacugugcgugugacagcggcugaucugugccugggcagcgcgaccc

SEQ ID NO: 42 - RNA, 23 nt, human
uuaaugcuaaucgugauaggggu

SEQ ID NO: 43 - RNA, 65 nt, human
cuguuaaugcuaaucgugauagggguuuuugccuccaacugacuccuacauauuagcauuaacag

SEQ ID NO: 44 - RNA, 22 nt, human
gagugugcuagaguccucgaag

SEQ ID NO: 45 - RNA, 64 nt, human
ucaucgaggcuagagucacgcuuggguaucggcuauugccugagugugcuagaguccucgaaga

SEQ ID NO: 46 - RNA, 19 nt, human
cauagcgagaccccgucug

SEQ ID NO: 47 - RNA, 73 nt, human
cagcugaacugggaggaucgcuugagcccagggguuugagaccagccugggcaacauagcgagaccccgucug

SEQ ID NO: 48 - RNA, 21 nt, human
ugagugugcuagaguccucga

SEQ ID NO: 49 - RNA, 58 nt, human
ucgaggcuagagucacgcuuggguaucggcuauugccugagugugcuagaguccucga

SEQ ID NO: 50 - RNA, 26 nt, human
guuggaggaugaaaguacggagugau

SEQ ID NO: 51 - RNA, 21 nt, human
ugagaugaagcacuguagcuc

SEQ ID NO: 52 - RNA, 106 nt, human
gcgcagcgcccugucucccagccugaggugcagugcugcaucucuggucaguugggagucugagaugaagcacuguagcucaggaagagagaaguuguucugcagc

SEQ ID NO: 53 - RNA, 23 nt, human
cccaguguuuagacuaucuguuc

SEQ ID NO: 54 - RNA, 23 nt, human
agcuacauugucugcuggguuuc

SEQ ID NO: 55 - RNA, 22 nt, human
ucucccaacccuuguaccagug

SEQ ID NO: 56 - RNA, 23 nt, human
aacauucaacgcugucggugagu

SEQ ID NO: 57 - RNA, 22 nt, human
ugagguaguagguuguaugguu

SEQ ID NO: 58 - RNA, 22 nt, human
ugggucuuugcgggcgagauga

SEQ ID NO: 59 - RNA, 21 nt, human
uucacaguggcuaaguucugc

SEQ ID NO: 60 - RNA, 23 nt, human
ucccuguccuccaggagcucacg

SEQ ID NO: 61 - RNA, 110 nt, human
ccagaggacaccuccacuccgucuacccaguguuuagacuaucuguucaggacucccaaauuguacaguagucugcacauugguuaggcugggcuggguuagacccucgg

SEQ ID NO: 62 - RNA, 110 nt, human
ugaacauccaggucuggggcaugaaccuggcauacaauguagauuucuguguucguuaggcaacagcuacauugucugcuggguuucaggcuaccuggaaacauguucuc

SEQ ID NO: 63 - RNA, 84 nt, human
cuccccauggcccugucucccaacccuuguaccagugcugggcucagacccugguacaggccugggggacagggaccuggggac

SEQ ID NO: 64 - RNA, 110 nt, human
agaagggcuaucaggccagccuucagaggacuccaaggaacauucaacgcugucggugaguuugggauuugaaaaaaccacugaccguugacuguaccuugggguccuua

SEQ ID NO: 65 - RNA, 110 nt, human
ugaguuuugagguugcuucagugaacauucaacgcugucggugaguuuggaauuaaaaucaaaaccaucgaccguugauuguacccuauggcuaaccaucaucuacucca

SEQ ID NO: 66 - RNA, 84 nt, human
gcauccggguugagguaguagguuguaugguuuagaguuacacccugggaguuaacuguacaaccuucuagcuuuccuuggagc

SEQ ID NO: 67 - RNA, 88 nt, human
cgaggaugggagcugagggcugggucuuugcgggcgagaugagggugucggaucaacuggccuacaaagucccaguucucggcccccg

SEQ ID NO: 68 - RNA, 97 nt, human
accucucuaacaaggugcagagcuuagcugauuggugaacagugauugguuuccgcuuuguucacaguggcuaaguucugcaccugaagagaaggug

SEQ ID NO: 69 - RNA, 94 nt, human
cggggcggccgcucucccuguccuccaggagcucacgugugccugccugugagcgccucgacgacagagccggcgccugccccagugucugcgc

SEQ ID NO: 70 - DNA, 28 bp, Artificial Sequence
  Synthetic
cagtcatttgggtgaggtagtaggttgt

SEQ ID NO: 71 - DNA, 28 bp, Artificial Sequence
  Synthetic
cagtcatttgggaaaccgttaccattac

SEQ ID NO: 72 - DNA, 28 bp, Artificial Sequence
  Synthetic
cagtcatttgggaacattcaacgctgtc

SEQ ID NO: 73 - DNA, 28 bp, Artificial Sequence
  Synthetic
cagtcatttgggaacccgtagatccgat

SEQ ID NO: 74 - DNA, 28 bp, Artificial Sequence
  Synthetic
cagtcatttggctctcccaacccttgta

SEQ ID NO: 75 - DNA, 28 bp, Artificial Sequence
  Synthetic
cagtcatttggctccctgtcctccagga

SEQ ID NO: 76 - DNA, 28 bp, Artificial Sequence
  Synthetic
cagtcatttggctgaccgatttctcctg

SEQ ID NO: 77 - DNA, 28 bp, Artificial Sequence
  Synthetic
cagtcatttggccccagtgtttagacta

SEQ ID NO: 78 - DNA, 28 bp, Artificial Sequence
  Synthetic
cagtcatttgggctgtgcgtgtgacagc

SEQ ID NO: 79 - DNA, 28 bp, Artificial Sequence
  Synthetic
cagtcatttggctgggtctttgcgggcg

SEQ ID NO: 80 - DNA, 28 bp, Artificial Sequence
  Synthetic
cagtcatttgggagctacattgtctgct

SEQ ID NO: 81 - DNA, 28 bp, Artificial Sequence
  Synthetic
cagtcatttgggttcacagtggctaagt

SEQ ID NO: 82 - DNA, 23 bp, Artificial Sequence
  Synthetic
ccgttttttttttttaaccatac

SEQ ID NO: 83 - DNA, 23 bp, Artificial Sequence
  Synthetic
ccgttttttttttttaactcagt

SEQ ID NO: 84 - DNA, 23 bp, Artificial Sequence
  Synthetic
ccgttttttttttttactcaccg

SEQ ID NO: 85 - DNA, 23 bp, Artificial Sequence
  Synthetic
ccgttttttttttttcacaagat

SEQ ID NO: 86 - DNA, 23 bp, Artificial Sequence
  Synthetic
ccgttttttttttttcactggta

SEQ ID NO: 87 - DNA, 23 bp, Artificial Sequence
  Synthetic
ccgttttttttttttcgtgagct

SEQ ID NO: 88 - DNA, 23 bp, Artificial Sequence
  Synthetic
ccgttttttttttttgaacacca

SEQ ID NO: 89 - DNA, 23 bp, Artificial Sequence
  Synthetic
ccgttttttttttttgaacagat

SEQ ID NO: 90 - DNA, 22 bp, Artificial Sequence
  Synthetic
cgttttttttttttcagccgct

SEQ ID NO: 91 - DNA, 22 bp, Artificial Sequence
  Synthetic
cgttttttttttttcatctcgc

SEQ ID NO: 92 - DNA, 22 bp, Artificial Sequence
  Synthetic
cgttttttttttttgaaaccca

SEQ ID NO: 93 - DNA, 22 bp, Artificial Sequence
  Synthetic
cgttttttttttttgcagaact

SEQ ID NO: 94 - DNA, 22 bp, Artificial Sequence
  Synthetic
gcgagcacagaattaatacgac

SEQ ID NO: 95 - DNA, 54 bp, Artificial Sequence
  Synthetic
gtcgtatccagtgcagggtccgaggtattcgcactggatacgacgaacaccagg

SEQ ID NO: 96 - DNA, 17 bp, Artificial Sequence
  Synthetic
tgaccgatttctcctgg

SEQ ID NO: 97 - DNA, 16 bp, Artificial Sequence
  Synthetic
gtgcagggtccgaggt

SEQ ID NO: 98 - DNA, 488 bp, human
tggataacaatgtattgggtgtcgattgtcatggggcaggggagagggcagtacacctggaggaccattttgtccacatcgacaccatcagtctgctcttagaggatgccctggagtattcggcgttgattgcggggcacccgaaatcagacttgccacctggactgtcgaggtgcagaccctgggagcaccactggcccatctcttacacaggctgaccgatttctcctggtgttcagagtctgtttttgtctagcaccatttgaaatcggttatgatgtagggggaaaagcagcagcctcgaagcctcatgccaactctgggcagcagcagcctgtggtttcctggaagatggatgggcagagaatagggaaggaagatcatgcttttccctactaacttctgtaactgcatgtatgatacattattgcagaggtaagagatagtttaatggatttttaaaaacaaattactataatttatctgat

(19) United States
(12) Patent Application Publication
(10) Pub. No.: US 2011/0071215 A1
(43) Pub. Date: Mar. 24, 2011

(54) COMPOSITIONS AND METHODS FOR DIAGNOSIS, PROGNOSIS AND TREATMENT OF MESOTHELIOMA

(76) Inventors: Harvey Pass, Bronxville, NY (US); Hila Benjamin, Gani Yohanan (IL); Moshe Hoshen, Jerusalem (IL); Sergey Ivanov, Brentwood, TN (US)

(21) Appl. No.: 12/995,409

(22) PCT Filed: May 26, 2009
(86) PCT No.: PCT/IL09/00524

§ 371 Date: Nov. 30, 2010

(30) Foreign Application Priority Data
Jun. 2, 2008 (US) 61/057882

(51) Int. Cl. A61K 31/7105; G01N 33/48; C12Q 1/68; A61K 31/711; A61P 35/00

(52) U.S. Cl. 514/44 R; 436/94; 435/6

(57) ABSTRACT

Described herein are compositions and methods for the diagnosis and treatment of mesothelioma patients and the prognosis of mesothelioma patients after surgical operation. Specifically the invention relates to microRNA molecules associated with diagnosis, treatment and prognosis of mesothelioma, as well as various nucleic acid molecules relating thereto or derived therefrom.

### CROSS REFERENCE TO RELATED APPLICATIONS

The present application claims priority under 35 U.S.C. §119(e) to U.S. Provisional Application No. 61/057,882, filed Jun. 2, 2008 which is herein incorporated by reference in its entirety.

### FIELD OF THE INVENTION

The invention relates to compositions and methods for diagnosis and treatment of mesothelioma and the prognosis of mesothelioma patients after surgical operation. Specifically the invention relates to microRNA molecules associated with the prognosis, diagnosis and treatment of mesothelioma, as well as various nucleic acid molecules relating thereto or derived therefrom.

### BACKGROUND OF THE INVENTION

In recent years, microRNAs (miRs, miRNAs) have emerged as an important novel class of regulatory RNA, which has profound impact on a wide array of biological processes. These small (typically 18-24 nucleotides long) non-coding RNA molecules can modulate protein expression patterns by promoting RNA degradation, inhibiting mRNA translation, and also affecting gene transcription. miRs play pivotal roles in diverse processes such as development and differentiation, control of cell proliferation, stress response and metabolism. There are currently about 900 known human miRs. The expression of many miRs was found to be altered in numerous types of human cancer, and in some cases strong evidence has been put forward in support of the conjecture that such alterations may play a causative role in tumor progression.

Mesothelioma is a tumor that occurs in the mesothelium that covers the surface of the pleura, peritoneum and pericardium that respectively envelop the organs of the chest cavity such as the lungs and heart, and abdominal organs such as the digestive tract and liver. In the case of diffuse pleural mesothelioma, chest pain is caused by invasion of the intercostal nerves on the side of the chest wall pleura, and respiratory and circulatory disorders may occur due to tumor growth and accumulation of pleural fluid in the pleura on the organ side (Takagi, Journal of Clinical and Experimental Medicine, (March Supplement), “Respiratory Diseases”, pp. 469-472, 1999). There is eventually proliferation into the adjacent mediastinal organs, progressing to direct invasion of the heart or development into the abdominal cavity by means of the diaphragm, or there may be development outside the chest cavity as a result of additional lymphatic or circulatory metastasis.

In the U.S., diffuse pleural mesothelioma is reported to occur in 3,000 persons annually, the number of cases began to increase prominently in the 1980's, and is frequently observed in men in their sixties, with the incidence in men being roughly five times that in women. According to recent reports in the U.S. and Europe, the incidence of mesothelioma is demonstrating a rapidly increasing trend, and based on epidemiological statistics from the U.K. in 1995, the number of deaths from mesothelioma is predicted to continue to increase over the next 25 years, and in the worst possible scenario, has been indicated as having a risk to the extent of accounting for 1% of all deaths among men born in the 1940's. Numerous different classifications of the clinical disease stages have been used for mesothelioma, and since the methods for classifying the disease stage used differ, previous therapeutic reports on mesothelioma have encountered difficulties when comparing the results of treatment (Nakano, Respiration, Vol. 18, No. 9, pp. 916-925, 1999). In addition, malignant mesothelioma has a causative relationship with exposure to asbestos, and this has also been demonstrated in animal experiments (Tada, Journal of Clinical and Experimental Medicine (March Supplement), “Respiratory Diseases”, pp. 406-408, 1999). Asbestos that has been inhaled into the respiratory tract reaches a location directly beneath the pleura where a tumor eventually develops due to chronic irritation for typically 20 years, and this tumor spreads in a thin layer over the entire surface of the pleura. Consequently, although malignant mesothelioma is classified as an asbestos-related disease, not all malignant mesothelioma is caused by asbestos, and well-documented exposure is only observed in about half of all patients. Malignant pleural mesothelioma is resistant to treatment, associated with an extremely poor prognosis, and requires that countermeasures be taken immediately (Nakano, Respiration, Vol. 18, No. 9, pp. 916-925, 1999).

The prognosis for malignant mesothelioma is influenced by the stage of the disease. Surgery, when performed as part of a multimodality therapy with cytotoxic chemotherapy and radiation therapy, as well as adjuvant immunological treatments (e.g., interferon or interleukin) can be effective treatments, but only in the rare event of an early stage diagnosis.

When dealing with the possibility of a mesothelioma in the pleura or the peritoneum few differential indications should be considered. Both the pleura and the peritoneum can have secondary malignancies with primaries at different rates, hence differentiation between mesothelioma and secondary malignancy or another primary from different source is important.

Much emphasis has been placed on the discovery and characterization of a unique tumor marker. However, no marker has been identified that has adequate sensitivity or specificity to, be clinically useful, although a combination of multiple markers has been shown to increase diagnostic accuracy. There is an unmet need for specific and accurate markers associated with mesothelioma, including those which would have prognostic significance in order to determine the extent of therapy necessary or reasonable for survival.

### SUMMARY OF THE INVENTION

According to some aspects of the present invention, specific nucleic acid sequences may be used for diagnosis, treatment and determination of the prognosis of mesothelioma.

According to one aspect of the invention, a method for determining a prognosis for mesothelioma in a subject is provided, the method comprising:
(a) obtaining a biological sample from the subject;
(b) determining the expression level in said sample of a nucleic acid sequence selected from the group consisting of SEQ ID NOS: 1-14, 38-41, 50, 53-55, 61-63 and sequences at least about 80% identical thereto from said sample; and
(c) comparing said expression level to a threshold expression level,
   wherein the comparison of the expression level of said nucleic acid to said threshold expression level is indicative of the prognosis of said subject.

According to one embodiment, an expression level of a nucleic acid sequence selected from the group consisting of SEQ ID NOS: 5-6, 11-12 and sequences at least about 80% identical thereto, above said threshold expression level, is indicative of poor prognosis in said subject.

According to another embodiment, an expression level of a nucleic acid sequence selected from the group consisting of SEQ ID NOS: 1-2, 8, 50 and sequences at least about 80% identical thereto, below said threshold expression level, is indicative of poor prognosis in said subject.

In some embodiments the prognosis is to predict overall survival in the subject. In other embodiments the prognosis is to predict the progression of mesothelioma in the subject.

In certain embodiments, the subject is a human.

In certain embodiments, the method is used to determine a course of treatment of the subject.

According to another aspect of the invention, a method for the diagnosis of mesothelioma in a subject is provided, the method comprising:
(a) obtaining a biological sample from the subject;
(b) determining the expression level in said sample of a nucleic acid sequence selected from the group consisting of SEQ ID NOS: 11-49, 51-52 and sequences at least about 80% identical thereto; and
(c) comparing said expression level to a control expression level,
wherein said determined expression level of said nucleic acids compared to said control expression level is indicative of the mesothelioma diagnosis of said subject.

According to one embodiment, an expression level of a nucleic acid sequence selected from the group consisting of SEQ ID NOS: 11-12, 34-35, 40-41, 46-47 and sequences at least about 80% identical thereto above said control expression level, is indicative of mesothelioma in said subject. According to another embodiment an expression level of a nucleic acid sequence selected from the group consisting of SEQ ID NOS: 13-33, 36-39, 51-52 and sequences at least about 80% identical thereto, below said control expression level, is indicative of mesothelioma in said subject.

According to another embodiment the biological sample is a tumor tissue at a specific stage.

In certain embodiments the biological sample obtained from the subject is selected from the group consisting of bodily fluid, a cell line and a tissue sample. In certain embodiments the tissue is a fresh, frozen, fixed, wax-embedded or formalin fixed paraffin-embedded (FFPE) tissue. In certain embodiments the tissue is mesothelium. In other embodiments the bodily fluid is serum.

According to some embodiments, the expression levels are determined by a method selected from the group consisting of nucleic acid hybridization, nucleic acid amplification, and a combination thereof. According to some embodiments, the nucleic acid hybridization is performed using a solid-phase nucleic acid biochip array or in situ hybridization.

According to other embodiments, the nucleic acid amplification method is real-time PCR. According to one embodiment, said real-time PCR is quantitative real-time PCR (qRT-PCR).

According to some embodiments, the RT-PCR method comprises forward and reverse primers. According to some embodiments, the forward primer comprises a sequence selected from the group consisting of SEQ ID NOS: 70-81 and sequences at least about 80% identical thereto.

According to some embodiments, the real-time PCR method further comprises hybridization with a probe. According to some embodiments, the probe comprises a sequence selected from the group consisting of SEQ ID NOS: 82-93 and sequences at least about 80% identical thereto.

The invention further provides a kit for prognosis of mesothelioma, said kit comprises a probe comprising a nucleic acid sequence that is complementary to a sequence selected from SEQ ID NOS: 1-14, 38-41, 50, 53-55, 61-63; to a fragment thereof, or to a sequence at least about 80% identical thereto. According to other embodiments the probe comprising a nucleic acid sequence selected from the group consisting of SEQ ID NOS: 82-93, and sequences having at least about 80% identity thereto.

The invention further provides a kit for diagnosis of mesothelioma, said kit comprises a probe comprising a nucleic acid sequence that is complementary to a sequence selected from SEQ ID NOS: 11-49, 51-52, to a fragment thereof, or to a sequence at least about 80% identical thereto.

According to other embodiments, the kit further comprises forward and reverse primers. According to some embodiments, said kit comprises reagents for performing in situ hybridization analysis.

Further provided in accordance with the invention is a method of treating or preventing mesothelioma in a subject in need thereof comprising administering to the subject an effective amount of a composition comprising a nucleic acid sequence selected from the group consisting of:
(a) SEQ ID NOS: 1-2, 98 and
(b) sequences at least about 80% identical to (a).

An additional aspect of the invention is a use of an effective amount of a composition comprising a nucleic acid sequence selected from the group consisting of:
(a) SEQ ID NOS: 1-2, 98
(b) sequences at least about 80% identical to (a),
   in the preparation of a medicament suitable for administration to a subject for the treatment or prevention of mesothelioma in said subject.

According to some embodiments the composition is suitable for administration in combination with at least one other anticancer agent in unit dosage form. According to some embodiments the anticancer agent is selected from the group consisting of cisplatin, pemetrexed, navelbine, gemcitibine, carboplatin, camptothecins, doxorubicin, cyclophosphamide, etoposide, vinblastine, Actinomycin D and cloposide.

### BRIEF DESCRIPTION OF THE DRAWINGS

FIGS. 1A-1D show Kaplan Meier plots, which correct for patients who were censored and calculates the significance of separation using log ranks. Data is presented for survival of mesothelioma patients, after surgical operation, by the following individual miRs: hsa-miR-29c* (SEQ ID NO: 1) (FIG. 1A), hsa-let-7b (SEQ ID NO: 5) (FIG. 1B), hsa-miR-768-5p (SEQ ID NO: 50) (FIG. 1C), hsa-miR-21* (SEQ ID NO: 11) (FIG. 1D), wherein the y-axis depicts fraction of survival and the X-axis depicts months of survival, with the solid line representing the lowest scoring tertile (12 subjects) and the dashed line representing the highest scoring tertile (13 subjects).

FIG. 2 shows a Kaplan Meier plot in which data is presented for survival of mesothelioma patients after surgical operation, using scores from the combined expression levels of miRs hsa-miR-29c* (SEQ ID NO: 1), hsa-miR-21* (SEQ ID NO: 11) and hsa-miR-34a (SEQ ID NO: 9). The Y-axis depicts the fraction of survival and the X-axis depicts months of survival, with the solid line representing the highest scoring tertile (12 subjects) and the dashed line depicting the lowest scoring tertile (13 subjects).

FIG. 3 shows a Kaplan Meier plot for survival of mesothelioma patients after surgical operation which was created using a combined model of the mesothelioma stage and the expression level of the following miRs: hsa-miR-29c* (SEQ ID NO: 1), hsa-miR-320 (SEQ ID NO: 3), hsa-let-7b (SEQ ID NO: 5) and hsa-miR-768-3p (SEQ ID NO: 7). The separation is much better than for each parameter alone. The Y-axis depicts fraction of survival and the X-axis depicts months of survival, with the solid line representing the highest scoring tertile (12 subjects) and the dashed line depicting the lowest scoring tertile (13 subjects).

FIGS. 4A-4D show scatter plots for 4 miRNAs that were differentially expressed between mesothelioma tumor samples and normal peritoneum samples, particularly hsa-miR-451 (SEQ ID NO: 13) (FIG. 4A), hsa-miR-486-5p (SEQ ID NO: 17) (FIG. 4B), hsa-miR-21 (SEQ ID NO: 34) (FIG. 4C) and hsa-miR-210 (SEQ ID NO: 40) (FIG. 4D). Each “+” represents a patient; the X-axis depicts the expression level of the miR in a tumor sample and the Y-axis depicts the expression level of the miR in the normal peritoneum sample from the same patient. The diagonal lines represent the expected expression for non-differentially expressed miRNAs (same expression level in tumor and normal peritoneum samples), and fold 2 factor lines.

FIG. 5 shows a scatter plot of average miR expression (in log(fluorescence)), for the 20 matched mesothelioma tumor sample/normal peritoneum sample pairs. The X-axis represents the median expression in tumor samples and the Y-axis represents the median expression in normal samples. The circled symbols relate to significantly differentially expressed miRs as determined by paired t-test, adjusted by the Benjamini and Hochberg False Discovery Rate of 0.01, including hsa-miR-133a (SEQ ID NO:23), hsa-miR-20b (SEQ ID NO: 32), hsa-miR-551b (SEQ ID NO: 30), hsa-miR-133b (SEQ ID NO: 28), hsa-miR-139-5p (SEQ ID NO: 26), hsa-miR-223 (SEQ ID NO: 21), hsa-mir-486-5p (SEQ ID NO: 17), hsa-miR-565 (SEQ ID NO: 19), hsa-miR-126 (SEQ ID NO: 15), hsa-miR-451 (SEQ ID NO: 13), hsa-miR-99a (SEQ ID NO: 38), hsa-miR-145 (SEQ ID NO: 36), hsa-miR-143 (SEQ ID NO: 51), hsa-miR-21* (SEQ ID NO: 11), hsa-miR-21 (SEQ ID NO: 34), hsa-miR-210 (SEQ ID NO: 40) and gam11 (SEQ ID NO: 46).

FIG. 6 shows a scatter plot of −log 10 (p-value) in paired t-test (x-axis) vs. absolute fold change (y-axis), per miR. The dark “+” symbols represent significantly differentially expressed miRs, as determined by paired t-test adjusted by the Benjamini and Hochberg False Discovery Rate of 0.01. The marked miRs, hsa-miR-133a (SEQ ID NO: 23), hsa-miR-133b (SEQ ID NO: 28), hsa-miR-223 (SEQ ID NO: 21), hsa-miR-126 (SEQ ID NO: 15), hsa-miR-155 (SEQ ID NO: 42), ambi_miR_{—}5893 (SEQ ID NO: 48), 70_{—}14 (SEQ ID NO: 44), hsa-miR-551b (SEQ ID NO: 30), hsa-miR-565 (SEQ ID NO: 19), hsa-miR-451 (SEQ ID NO: 13), miR-139-5p (SEQ ID NO: 26), hsa-miR-20b (SEQ ID NO: 32), hsa-miR-21 (SEQ ID NO: 34) and hsa-mir-486-5p (SEQ ID NO: 17), have both a statistically significant differential expression, and a high fold change (>2) between tumor and normal samples.

FIG. 7 shows a scatter plot of average miR expression (in log(fluorescence)) in the training set, as described in example 2.II.a. The X-axis represents the median expression in samples from patients with good prognosis (TTP (time to progression)>12 months, n=10) and the Y-axis represents the median expression in samples from patients with poor prognosis (TTP<12 months, n=24). As indicated, hsa-miR-29c* (SEQ ID NO: 1) had significantly different expression (p value 0.000355, fold change 1.8) after adjusting for the false discovery rate (FDR).

FIG. 8 shows a Kaplan Meier plot, in which data is presented for TTP (time to progression) of mesothelioma patients of the training set as described in example 2.II.a, by the expression of hsa-miR-29c* (SEQ ID NO: 1). The Y-axis depicts fraction of progression-free patients and the X-axis depicts months of progression-free survival. The dashed curve represents the group (n=19) with expression more than 8.8, having a median progression-free survival of 4 months, and the solid curve represents the group (n=18) with expression equal to or less than 8.8, having a median progression-free survival of 14 months.

FIG. 9 show Kaplan Meier plots, in which data is presented for TTP (time to progression) of mesothelioma patients of the training set as described in example 2.II.a, by the expression of hsa-miR-29c* (SEQ ID NO: 1) in patients from various stages of mesothelioma. The Y-axis depicts fraction of progression-free patients and the X-axis depicts months of progression-free survival.

FIG. 9A refers to stage I/II, in which the dashed curve represents the group (n=5) with expression more than 8.8, and the solid curve represents the group (n=5) with expression equal to or less than 8.8.

FIG. 9B refers to stage II/IV, in which the dashed curve represents the group (n=14) with expression more than 8.8 and the solid curve represents the group (n=13) with expression equal to or less than 8.8.

FIG. 10 shows a Kaplan Meier plot, in which data is presented for survival of mesothelioma patients of the training set as described in example 2.II.b, by the expression of hsa-miR-29c* (SEQ ID NO: 1). The Y-axis depicts the fraction of surviving patients and the X-axis depicts months of survival. The solid curve represents the group (n=18) with expression equal to or less than 8.8, having a median survival of 8 months, and the dashed curve represents the group (n=19) with expression more than 8.8, having a median of survival of 32 months.

FIG. 11 shows a Kaplan Meier plot, in which data is presented for TTP (time to progression) of mesothelioma patients of the test set as described in example 2.III.a, by the expression of hsa-miR-29c* (SEQ ID NO: 1). The Y-axis depicts the fraction of progression-free patients and the X-axis depicts months of progression-free survival. The solid curve represents the group (n=35) with expression equal to or less than 8.8, having a median survival of 5.5 months, and the dashed curve represents the group (n=57) with expression more than 8.8, having a median survival of 12.8 months.

FIG. 12 shows a Kaplan Meier plot, in which data is presented for survival of mesothelioma patients of the test set as described in example 2.III.b, by the expression of hsa-miR-29c* (SEQ ID NO: 1). The Y-axis depicts the fraction of surviving patients and the X-axis depicts months of survival. The solid curve represents the group (n=35) with expression equal to or less than 8.8, having a median of survival of 9.1 months, and the dashed curve represents the group (n=57) with expression more than 8.8, having a median survival of 21.6 months.

FIG. 13 shows a Kaplan Meier plot, in which data is presented for survival of epithelial mesothelioma patients of the test set as described in example 2.III.b, by the expression of hsa-miR-29c* (SEQ ID NO: 1). The Y-axis depicts the fraction of surviving patients and the X-axis depicts months of survival. The solid curve represents the group (n=14) with expression equal to or less than 8.8, having a median of survival of 9.1 months, and the dashed curve represents the group (n=44) with expression more than 8.8, having a median of survival of 21.6 months.

FIGS. 14A-14B show dot-plot presentations of the expression of hsa-miR-29c* (SEQ ID NO: 1). Left columns: patients with good prognosis. Right column: patients with poor prognosis. Horizontal lines mark group median.

FIG. 14A is based on microarray results. The Y-axis depicts log₂ of normalized miR expression

FIG. 14B is based on qRT-PCR results. The Y-axis depicts 50-Cₜ.

FIG. 15 shows a Kaplan Meier plot in which PCR data is presented for survival of 16 mesothelioma patients as described in example 2.1V, by the expression of hsa-miR-29c* (SEQ ID NO: 1). The Y-axis depicts the fraction of surviving patients and the X-axis depicts months of survival. The solid curve represents the group (n=9) with a 50-Cₜ measurement equal to or less than 16.8, having a median of survival of 2.8 months, and the dashed curve represents the group (n=7) with a 50-Cₛ measurement of more than 16.8, having a median of survival of 35.4 months.

FIG. 16 demonstrates the validation of miR-29c* (SEQ ID NO: 1) delivery to Hmeso (left bars), HP-1 (middle bars) and H2595 (right bars) mesothelioma lines by stem-loop RT-PCR. The PCR specific product of miR-29c* (50 bp) was visualized by ethidium bromide staining in 4% MetaPhor agarose.

FIGS. 17A-17D are histograms depicting 112595 (left bars), HP-1 (middle bars) and Hmeso (right bars) mesothelioma lines. The white bars represent cells treated with lipofectamine only. The diagonal bars represent native cells (negative control cells) and the dotted bars represent cells transfected with miR-29c* (SEQ ID NO: 1) mimic (p<0.005).

In FIG. 17A the Y-axis depicts the fold change in cell density 48 hours following transfection, based on the results of cell proliferation assay; In FIG. 17B the Y-axis depicts number of cells invaded at 48 hours following transfection, based on the results of matrigel invasion assay; In FIG. 17C the Y-axis depicts percentage of closure at 48 hours following transfection, based on the results of scratch (wound healing) assay. In FIG. 17D the Y-axis depicts number of colonies 21 days following transfection based on soft agar colony formation assay.

FIG. 17E shows pictures of the colony formation assay 21 days following transfection of H2595 (upper pictures) and HP-1 (lower pictures). The left column represents cells treated with lipofectamine only; the middle column represents native cells (negative control cells) and the right column represents cells transfected with miR-29c* (SEQ ID NO: 1) mimic.

FIG. 18A shows the complete insert sequence of miR-29c* (SEQ ID NO: 98) with pre-miR hairpin (SEQ ID NO: 2) and the resulting mature miR-29c* sequence (SEQ ID NO: 1).

FIG. 18B shows the resulting stem loop structure of miR-29c* (SEQ ID NO: 2).

### DETAILED DESCRIPTION OF THE INVENTION

The invention is based in part on the discovery that miRNA expression can serve as a novel tool for the diagnosis, determination of prognosis and treatment of mesothelioma.

According to some aspects of the invention, specific nucleic acid sequences (SEQ ID NOS: 11-49, 51-52 and sequences at least about 80% identical thereto) may be used for the diagnosis of mesothelioma, and specific nucleic acids (SEQ ID NOS: 1-12 and 50, and sequences at least about 80% identical thereto) may be used for the determination of prognosis of mesothelioma.

According to other aspects of the invention, the presence of a single microRNA has significant prognostic implications for malignant pleural mesothelioma (MM). Using a proprietary microarray capable of analyzing over 900 miRs, hsa-miR-29c* (SEQ ID NO: 1) was not only able separate MM patients by time to progression after surgery, but also stratified these patients by their survival. The microRNA is preferentially expressed in epithelial MM, and further stratifies this group of mesotheliomas which are associated with a better prognosis into favorable and unfavorable cohorts. Validation of mir29c* was accomplished using RT-PCR, and in vitro functional assays in which the miR is expressed in MM cell lines are totally consistent with the clinical findings that the expression of this miR decreases the tumors proliferative, migration and invasive potential. There are no reports in the literature as of yet with regard to the function of this member of the 29c family.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9 and 7.0 are explicitly contemplated.

### 1. DEFINITIONS

About

As used herein, the term “about” refers to +/−10%.

Administering

“Administering” means providing a pharmaceutical agent or composition to a subject, and includes, but is not limited to, administering by a medical professional and self-administering.

“Parenteral administration,” means administration through injection or infusion. Parenteral administration includes, but is not limited to, subcutaneous administration, intravenous administration, or intramuscular administration.
“Subcutaneous administration” means administration just below the skin.
“Intravenous administration” means administration into a vein.
“Intratumoral administration” means administration within a tumor.
“Chemoembolization” means a procedure in which the blood supply to a tumor is blocked surgically or mechanically and chemotherapeutic agents are administered directly into the tumor.
“Intracavitary administration” means administration directly into a cavity with suspected or diagnosed tumor with said cavity represented as either left or right pleural cavity or the abdominal cavity or the pericardial space.

Amelioration

Amelioration as used herein, refers to a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

Antisense

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

Attached

“Attached” or “immobilized” as used herein refer to a probe and a solid support and may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe, or both. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

Biological Sample

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, FFPE samples, frozen sections taken for histological purposes, blood, plasma, serum, sputum, stool, tears, mucus, hair, and skin. Biological samples also include explants and primary and/or transformed cell cultures derived from animal or patient tissues.

Biological samples may also be blood, a blood fraction, urine, effusions, ascitic fluid, saliva, cerebrospinal fluid, cervical secretions, vaginal secretions, endometrial secretions, gastrointestinal secretions, bronchial secretions, sputum, cell line, tissue sample, cellular content of fine needle aspiration (FNA) or secretions from the breast. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

Cancer

The term “cancer” is meant to include all types of cancerous growths or oncogenic processes, metastatic tissues or malignantly transformed cells, tissues, or organs, irrespective of histopathologic type or stage of invasiveness. Examples of cancers include but are nor limited to solid tumors and leukemias, including: glioblastoma, apudoma, choristoma, branchioma, malignant carcinoid syndrome, carcinoid heart disease, carcinoma (e.g., Walker, basal cell, basosquamous, Brown-Pearce, ductal, Ehrlich tumor, small cell lung, non-small cell lung (e.g., lung squamous cell carcinoma, lung adenocarcinoma and lung undifferentiated large cell carcinoma), oat cell, papillary, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, mesothelioma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental; Kaposi, and mast cell), neurofibromatosis, and cervical dysplasia, and other conditions in which cells have become immortalized or transformed.

Cancer Prognosis

A forecast or prediction of the probable course or outcome of the cancer and response to its treatment. As used herein, cancer prognosis includes distinguishing between cancer stages and subtypes, and the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

Chemotherapeutic Agent

A drug used to treat a disease, especially cancer. In relation to cancer the drugs typically target rapidly dividing cells, such as cancer cells. Non-limiting examples of chemotherapeutic agents include cisplatin, carboplatin, camptothecins, doxorubicin, cyclophosphamide, paclitaxel, etoposide, vinblastine, Actinomycin D and cloposide.

Classification

“Classification” as used herein refers to a procedure and/or algorithm in which individual items are placed into groups or classes based on quantitative information on one or more characteristics inherent in the items (referred to as traits, variables, characters, features, etc) and based on a statistical model and/or a training set of previously labeled items. According to one embodiment, classification means determination of the type of cancer.

Complement

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

Ct

Ct signals represent the first cycle of PCR where amplification crosses a threshold (cycle threshold) of fluorescence. Accordingly, low values of Ct represent high abundance or expression levels of the microRNA.

In some embodiments the PCR Ct signal is normalized such that the normalized Ct remains inversed from the expression level. In other embodiments the PCR Ct signal may be normalized and then inverted such that low normalized-inverted Ct represents low abundance or expression levels of the microRNA.

Detection

“Detection” means detecting the presence of a component in a sample. Detection also means detecting the absence of a component. Detection also means measuring the level of a component, either quantitatively or qualitatively.

Differential Expression

“Differential expression” means qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated-resulting in an increased amount of transcript, or down-regulated-resulting in a decreased amount of transcript. The degree to which expression differs need only be large enough to quantify via standard characterization techniques such as expression arrays, quantitative reverse transcriptase PCR, northern analysis, real-time PCR, in situ hybridization and RNase protection.

Dose

“Dose” as used herein means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

Dosage Unit

“Dosage unit” as used herein means a form in which a pharmaceutical agent is provided. In certain embodiments, a dosage unit is a vial containing lyophilized oligonucleotide. In certain embodiments, a dosage unit is a vial containing reconstituted oligonucleotide.

Expression Profile

“Expression profile” as used herein may mean a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art. Nucleic acid sequences of interest are nucleic acid sequences that are found to be predictive, including the nucleic acid sequences provided above, where the expression profile may include expression data for 5, 10, 20, 25, 50, 100 or more of, including all of the listed nucleic acid sequences. The term “expression profile” may also mean measuring the abundance of the nucleic acid sequences in the measured samples.

Expression Ratio

“Expression ratio” as used herein refers to relative expression levels of two or more nucleic acids as determined by detecting the relative expression levels of the corresponding nucleic acids in a biological sample.

FDR

When performing multiple statistical tests, for example in comparing the signal between two groups in multiple data features, there is an increasingly high probability of obtaining false positive results, by random differences between the groups that can reach levels that would otherwise be considered as statistically significant. In order to limit the proportion of such false discoveries, statistical significance is defined only for data features in which the differences reached a p-value (by two-sided t-test) below a threshold, which is dependent on the number of tests performed and the distribution of p-values obtained in these tests.

Fragment

“Fragment” is used herein to indicate a non-full length part of a nucleic acid or polypeptide. Thus, a fragment is itself also a nucleic acid or polypeptide, respectively.

Gene

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

Groove Binder/Minor Groove Binder (MGB)

“Groove binder” and/or “minor groove binder” may be used interchangeably and refer to small molecules that fit into the minor groove of double-stranded DNA, typically in a sequence-specific manner. Minor groove binders may be long, flat molecules that can adopt a crescent-like shape and thus, fit snugly into the minor groove of a double helix, often displacing water. Minor groove binding molecules may typically comprise several aromatic rings connected by bonds with torsional freedom such as furan, benzene, or pyrrole rings. Minor groove binders may be antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues, including those described in Nucleic Acids in Chemistry and Biology, 2d ed., Blackburn and Gait, eds., Oxford University Press, 1996, and PCT Published Application No. WO 03/078450, the contents of which are incorporated herein by reference. A minor groove binder may be a component of a primer, a probe, a hybridization tag complement, or combinations thereof. Minor groove binders may increase the Tₘ of the primer or a probe to which they are attached, allowing such primers or probes to effectively hybridize at higher temperatures.

Host Cell

“Host cell” as used herein may be a naturally occurring cell or a transformed cell that may contain a vector and may support replication of the vector.

Identity

“Identical” or “identity” as used herein in the context of two or more nucleic acids or polypeptide sequences mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

In Situ Detection

“In situ detection” as used herein means the detection of expression or expression levels in the original site hereby meaning in a tissue sample such as biopsy.

Inhibit

“Inhibit” as used herein may mean prevent, suppress, repress, reduce or eliminate.

Label

“Label” as used herein means a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

Logistic Regression

Logistic regression is part of a category of statistical models called generalized linear models. Logistic regression allows one to predict a discrete outcome, such as group membership, from a set of variables that may be continuous, discrete, dichotomous, or a mix of any of these. The dependent or response variable is dichotomous, for example, one of two possible types of cancer. Logistic regression models the natural log of the odds ratio, i.e. the ratio of the probability of belonging to the first group (P) over the probability of belonging to the second group (1-P), as a linear combination of the different expression levels (in log-space) and of other explaining variables. The logistic regression output can be used as a classifier by prescribing that a case or sample will be classified into the first type if P is greater than 0.5 or 50%. Alternatively, the calculated probability P can be used as a variable in other contexts such as a 1D or 2D threshold classifier.

1D/2D Threshold Classifier

“1D/2D threshold classifier” used herein may mean an algorithm for classifying a case or sample such as a cancer sample into one of two possible types such as two types of cancer or two types of prognosis (e.g. good and bad). For a 1D threshold classifier, the decision is based on one variable and one predetermined threshold value; the sample is assigned to one class if the variable exceeds the threshold and to the other class if the variable is less than the threshold. A 2D threshold classifier is an algorithm for classifying into one of two types based on the values of two variables. A score may be calculated as a function (usually a continuous function) of the two variables; the decision is then reached by comparing the score to the predetermined threshold, similar to the 1D threshold classifier.

Metastasis

“Metastasis” as used herein means the process by which cancer spreads from the place at which it first arose as a primary tumor to other locations in the body. The metastatic progression of a primary tumor reflects multiple stages, including dissociation from neighboring primary tumor cells, survival in the circulation, and growth in a secondary location.

Mismatch

“Mismatch” means a nucleobase of a first nucleic acid that is not capable of pairing with a nucleobase at a corresponding position of a second nucleic acid.

Modulation

“Modulation” as used herein means a perturbation of function or activity. In certain embodiments, modulation means an increase in gene expression. In certain embodiments, modulation means a decrease in gene expression.

Nucleic Acid

“Nucleic acid” or “oligonucleotide” or “polynucleotide” as used herein mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005) and Soutschek et al., Nature 432:173-178 (2004), which are incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver. Mixtures of naturally occurring nucleic acids and analogs may be made; alternatively, mixtures of different nucleic acid analogs, and mixtures of naturally occurring nucleic acids and analogs may be made.

Overall Survival Time

“Overall survival time” or “survival time”, as used herein means the time period for which a subject survives after diagnosis of or treatment for a disease. In certain embodiments, the disease is cancer.

Pharmaceutical Agent

Pharmaceutical agent as used herein means a substance that provides a therapeutic effect when administered to a subject. “Pharmaceutical composition” means a mixture of substances suitable for administering to an individual that includes a pharmaceutical agent. For example, a pharmaceutical composition may comprise a modified oligonucleotide and a sterile aqueous solution. “Active pharmaceutical ingredient” means the substance in a pharmaceutical composition that provides a desired effect.

Prevention

Prevention as used herein means delaying or forestalling the onset or development or progression of a condition or disease for a period of time, including weeks, months, or years.

Progression-Free Survival

“Progression-free survival” means the time period for which a subject having a disease survives, without the disease getting worse. In certain embodiments, progression-free survival is assessed by staging or scoring the disease. In certain embodiments, progression-free survival of a subject having cancer is assessed by evaluating tumor size, tumor number, and/or metastasis.

Probe

“Probe” as used herein means an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

Promoter

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

Reference Expression Profile

As used herein the term “reference expression profile” means a value that statistically correlates to a particular outcome when compared to an assay result. In preferred embodiments the reference value is determined from statistical analysis of studies that compare microRNA expression with known clinical outcomes. The reference value may be a threshold score value or a cutoff score value. Typically a reference value will be a threshold above which one outcome is more probable and below which an alternative threshold is more probable.

Sensitivity

“sensitivity” used herein may mean a statistical measure of how well a binary classification test correctly identifies a condition, for example how frequently it correctly classifies a cancer into the correct type out of two possible types. The sensitivity for class A is the proportion of cases that are determined to belong to class “A” by the test out of the cases that are in class “A”, as determined by some absolute or gold standard.

Specificity

“Specificity” used herein may mean a statistical measure of how well a binary classification test correctly identifies a condition, for example how frequently it correctly classifies a cancer into the correct type out of two possible types. The specificity for class A is the proportion of cases that are determined to belong to class “not A” by the test out of the cases that are in class “not A”, as determined by some absolute or gold standard.

Side Effect

Side effect as used herein means a physiological response attributable to a treatment other than desired effects.

Selectable Marker

“Selectable marker” as used herein means any gene which confers a phenotype on a host cell in which it is expressed to facilitate the identification and/or selection of cells which are transfected or transformed with a genetic construct. Representative examples of selectable markers include the ampicillin-resistance gene (Amp^{r}), tetracycline-resistance gene (Tc^{r}), bacterial kanamycin-resistance gene (Kan^{r}), zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene.

Stringent Hybridization Conditions

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium).

Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

Substantially Complementary

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

Substantially Identical

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

Subject

As used herein, the term “subject” refers to a human or non-human animal selected for treatment or therapy. The methods of the present invention are preferably applied to human subjects. “Subject in need thereof” refers to a subject identified as in need of a therapy or treatment. In certain embodiments, a subject is in need of treatment for mesothelioma. In such embodiments, a subject has one or more clinical indications of mesothelioma or is at risk for developing mesothelioma.

Target Nucleic Acid

“Target nucleic acid” as used herein means a nucleic acid or variant thereof that may be bound by another nucleic acid. A target nucleic acid may be a DNA sequence. The target nucleic acid may be RNA. The target nucleic acid may comprise a mRNA, tRNA, shRNA, siRNA or Piwi-interacting RNA, or a pri-miRNA, pre-miRNA, miRNA, or anti-miRNA.

The target nucleic acid may comprise a target miRNA binding site or a variant thereof. One or more probes may bind the target nucleic acid. The target binding site may comprise 5-100 or 10-60 nucleotides. The target binding site may comprise a total of 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 61, 62 or 63 nucleotides. The target site sequence may comprise at least 5 nucleotides of the sequence of a target miRNA binding site disclosed in U.S. patent application Ser. Nos. 11/384,049, 11/418,870 or 11/429,720, the contents of which are incorporated herein.

Therapy

“Therapy” as used herein means a disease treatment method. In certain embodiments, therapy includes, but is not limited to, tyrosine kinase inhibition therapy, chemotherapy, surgical resection, transplant, radiation therapy, “gene therapy”, immunotherapy, and/or chemoembolization. “Therapeutic agent” means a pharmaceutical agent used for the cure, amelioration or prevention of a disease. “Recommended therapy” means a treatment recommended by a medical professional for the treatment, amelioration, or prevention of a disease.

Therapeutically Effective Amount

“Therapeutically effective amount” or “therapeutically efficient” used herein as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the disease.

Threshold Expression Level

As used herein, the phrase “threshold expression level” refers to a reference expression value. Measured values are compared to a corresponding threshold expression level to determine the prognosis of a subject.

Tissue Sample

As used herein, a tissue sample is tissue obtained from a tissue biopsy using methods well known to those of ordinary skill in the related medical arts. The phrase “suspected of being cancerous” as used herein means a cancer tissue sample believed by one of ordinary skill in the medical arts to contain cancerous cells. Methods for obtaining the sample from the biopsy include gross apportioning of a mass, microdissection, laser-based microdissection, or other art-known cell-separation methods.

Treat

“Treat” or “treating” used herein when referring to protection of a subject from a condition may mean preventing, suppressing, repressing, or eliminating the condition. Preventing the condition involves administering a composition described herein to a subject prior to onset of the condition. Suppressing the condition involves administering the composition to a subject after induction of the condition but before its clinical appearance. Repressing the condition involves administering the composition to a subject after clinical appearance of the condition such that the condition is reduced or prevented from worsening. Elimination of the condition involves administering the composition to a subject after clinical appearance of the condition such that the subject no longer suffers from the condition.

Unit Dosage Form

“Unit dosage form,” used herein may refer to a physically discrete unit suitable as a unitary dosage for a human or animal subject. Each unit may contain a predetermined quantity of a composition described herein, calculated in an amount sufficient to produce a desired effect in association with a pharmaceutically acceptable diluent, carrier or vehicle. The specifications for a unit dosage form may depend on the particular composition employed and the effect to be achieved, and the pharmacodynamics associated with the composition in the host.

Variant

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

Vector

“Vector” as used herein means a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Wild Type

As used herein, the term “wild type” sequence refers to a coding, a non-coding or an interface sequence which is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

### 2. MICRORNAS And Their Processing

A gene coding for a microRNA (miRNA) may be transcribed leading to production of an miRNA precursor known as the pri-miRNA. The pri-miRNA may be part of a polycistronic RNA comprising multiple pri-miRNAs. The pri-miRNA may form a hairpin structure with a stem and loop. The stem may comprise mismatched bases.

The hairpin structure of the pri-miRNA may be recognized by Drosha, which is an RNase III endonuclease. Drosha may recognize terminal loops in the pri-miRNA and cleave approximately two helical turns into the stem to produce a 60-70 nucleotide precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of the stem (˜10 nucleotides) extending beyond the Drosha cleavage site may be essential for efficient processing. The pre-miRNA may then be actively transported from the nucleus to the cytoplasm by Ran-GTP and the export receptor Ex-portion-5.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. mRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repression or activation), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-7 of the miRNA. Only one case has been reported in animals where the interaction between the miRNA and its target was along the entire length of the miRNA. This was shown for mir-196 and Hox B8 and it was further shown that mir-196 mediates the cleavage of the Hox B8 mRNA (Yekta et al 2004, Science 304-594). Otherwise, such interactions are known only in plants (Bartel & Bartel 2003, Plant Physiol 132-709).

A number of studies have studied the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85).

Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet. 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and the binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

### 3. NUCLEIC ACIDS

Nucleic acids are provided herein. The nucleic acids comprise the sequence of SEQ ID NOS: 1-98, or variants thereof. The variant may be a complement of the referenced nucleotide sequence. The variant may also be a nucleotide sequence that is substantially identical to the referenced nucleotide sequence or the complement thereof. The variant may also be a nucleotide sequence which hybridizes under stringent conditions to the referenced nucleotide sequence, complements thereof, or nucleotide sequences substantially identical thereto.

The nucleic acid may have a length of from 10 to 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference.

3a. Nucleic Acid Complexes

The nucleic acid may further comprise one or more of the following: a peptide, a protein, a RNA-DNA hybrid, an antibody, an antibody fragment, a Fab fragment, and an aptamer.

3b. Pri-miRNA

The nucleic acid may comprise a sequence of a pri-miRNA or a variant thereof. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

The pri-miRNA may form a hairpin structure. The hairpin may comprise a first and a second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy of less than −25 Kcal/mole, as calculated by the Vienna algorithm, with default parameters as described in Hofacker et al., Monatshefte f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

3c. Pre-miRNA

The nucleic acid may also comprise a sequence of a pre-miRNA or a variant thereof. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-69, or variants thereof.

3d. miRNA

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1-69 or variants thereof.

3e. Anti-miRNA

The nucleic acid may also comprise a sequence of an anti-miRNA capable of blocking the activity of a miRNA or miRNA*, such as by binding to the pri-miRNA, pre-miRNA, miRNA or miRNA* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially identical or complimentary to the 5′ of a miRNA and at least 5-12 nucleotides that are substantially complimentary to the flanking regions of the target site from the 5′ end of the miRNA, or (b) at least 5-12 nucleotides that are substantially identical or complimentary to the 3′ of a miRNA and at least 5 nucleotide that are substantially complimentary to the flanking region of the target site from the 3′ end of the miRNA.

3f microRNA Binding Site of Target

The nucleic acid may also comprise a sequence of a target binding site or a variant thereof. The target site sequence may comprise a total of 5-100 or 10-60 nucleotides. The target site sequence may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 nucleotides.

### 4. SYNTHETIC GENE

A synthetic gene is also provided comprising a nucleic acid described herein operably linked to a transcriptional and/or translational regulatory sequence. The synthetic gene may be capable of modifying the expression of a target gene with a binding site for a nucleic acid described herein. Expression of the target gene may be modified in a cell, tissue or organ. The synthetic gene may be synthesized or derived from naturally-occurring genes by standard recombinant techniques. The synthetic gene may also comprise terminators at the 3′-end of the transcriptional unit of the synthetic gene sequence. The synthetic gene may also comprise a selectable marker.

### 5. VECTOR

A vector is also provided comprising a synthetic gene described herein. The vector may be an expression vector. An expression vector may comprise additional elements. For example, the expression vector may have two replication systems allowing it to be maintained in two organisms, e.g., in one host cell for expression and in a second host cell (e.g., bacteria) for cloning and amplification. For integrating expression vectors, the expression vector may contain at least one sequence homologous to the host cell genome, and preferably two homologous sequences which flank the expression construct. The integrating vector may be directed to a specific locus in the host cell by selecting the appropriate homologous sequence for inclusion in the vector. The vector may also comprise a selectable marker gene to allow the selection of transformed host cells.

### 6. HOST CELL

A host cell is also provided comprising a vector, synthetic gene or nucleic acid described herein. The cell may be a bacterial, fungal, plant, insect or animal cell. For example, the host cell line may be DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines may be available from commercial services, the American Tissue Culture Collection or from published literature.

### 7. PROBES

A probe is provided herein. A probe may comprise a nucleic acid. The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may comprise a nucleic acid of 18-25 nucleotides.

A probe may be capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. A probe may be single stranded or partially single and partially double stranded. The strandedness of the probe is dictated by the structure, composition, and properties of the target sequence. Probes may be directly labeled or indirectly labeled.

The probe may be a test probe. The test probe may comprise a nucleic acid sequence that is complementary to a miRNA, a miRNA*, a pre-miRNA, or a pri-miRNA.

The probe may further comprise a linker. The linker may be 10-60 nucleotides in length. The linker may be 20-27 nucleotides in length. The linker may be of sufficient length to allow the probe to be a total length of 45-60 nucleotides. The linker may not be capable of forming a stable secondary structure, or may not be capable of folding on itself, or may not be capable of folding on a non-linker portion of a nucleic acid contained in the probe. The sequence of the linker may not appear in the genome of the animal from which the probe non-linker nucleic acid is derived.

### 8. REVERSE TRANSCRIPTION

Target sequences of a cDNA may be generated by reverse transcription of the target RNA. Methods for generating cDNA may be reverse transcribing polyadenylated RNA or alternatively, RNA with a ligated adaptor sequence.

The RNA may be ligated to an adapter sequence prior to reverse transcription. A ligation reaction may be performed by T4 RNA ligase to ligate an adaptor sequence at the 3′ end of the RNA. Reverse transcription (RT) reaction may then be performed using a primer comprising a sequence that is complementary to the 3′ end of the adaptor sequence.

Polyadenylated RNA may be used in a reverse transcription (RT) reaction using a poly(T) primer comprising a 5′ adaptor sequence. The poly(T) sequence may comprise 8, 9, 10, 11, 12, 13, or 14 consecutive thymines.

The reverse transcript of the RNA may be amplified by real time PCR, using a specific forward primer comprising at least 15 nucleic acids complementary to the target nucleic acid and a 5′ tail sequence; a reverse primer that is complementary to the 3′ end of the adaptor sequence; and a probe comprising at least 8 nucleic acids complementary to the target nucleic acid. The probe may be partially complementary to the 5′ end of the adaptor sequence. The sequence of the probe may be selected from SEQ ID NOS: 82-93 and sequences at least about 80% identical thereto. The sequence of the forward primer may be selected from SEQ ID NO: 70-81 and sequences at least about 80% identical thereto. The sequence of the reverse primer may be SEQ ID NO: 94 or sequences at least about 80% identical thereto.

Methods of amplifying target nucleic acids are described herein. The amplification may be by a method comprising PCR. The first cycles of the PCR reaction may have an annealing temp of 56° C., 57° C., 58° C., 59° C., or 60° C. The first cycles may comprise 1-10 cycles. The remaining cycles of the PCR reaction may be 60° C. The remaining cycles may comprise 2-40 cycles. The annealing temperature may cause the PCR to be more sensitive. The PCR may generate longer products that can serve as higher stringency PCR templates.

The PCR reaction may comprise a forward primer. The forward primer may comprise 15, 16, 17, 18, 19, 20, or 21 nucleotides identical to the target nucleic acid.

The 3′ end of the forward primer may be sensitive to differences in sequence between a target nucleic acid and a sibling nucleic acid.

The forward primer may also comprise a 5′ overhanging tail. The 5′ tail may increase the melting temperature of the forward primer. The sequence of the 5′ tail may comprise a sequence that is non-identical to the genome of the animal from which the target nucleic acid is isolated. The sequence of the 5′ tail may also be synthetic. The 5′ tail may comprise 8, 9, 10, 11, 12, 13, 14, 15, or 16 nucleotides.

The PCR reaction may comprise a reverse primer. The reverse primer may be complementary to a target nucleic acid. The reverse primer may also comprise a sequence complementary to an adaptor sequence. The sequence complementary to an adaptor sequence may comprise 12-24 nucleotides.

### 9. BIOCHIP

A biochip is also provided. The biochip may comprise a solid substrate comprising an attached probe or plurality of probes described herein. The probes may be capable of hybridizing to a target sequence under stringent hybridization conditions. The probes may be attached at spatially defined locations on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrate materials include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as flexible foam, including closed cell foams made of particular plastics.

The substrate of the biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker.

The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

### 10. DIAGNOSTICS

A method of diagnosis is also provided. The method comprises detecting a differential expression level of mesothelioma-associated nucleic acids in a biological sample. The sample may be derived from a patient. Diagnosis of a cancer state, and its histological type, in a patient may allow for prognosis and selection of therapeutic strategy. Further, the developmental stage of cells may be classified by determining temporarily expressed cancer-associated nucleic acids.

In situ hybridization of labeled probes to tissue arrays may be performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

### 11. KITS

A kit is also provided and may comprise a nucleic acid described herein together with any or all of the following: assay reagents, buffers, probes and/or primers, and sterile saline or another pharmaceutically acceptable emulsion and suspension base. In addition, the kits may: include instructional materials containing directions (e.g., protocols) for the practice of the methods described herein.

For example, the kit may be used for the amplification, detection, identification or quantification of a target nucleic acid sequence. The kit may comprise a poly(T) primer, a forward primer, a reverse primer, and a probe.

Any of the compositions described herein may be comprised in a kit. In a non-limiting example, reagents for isolating miRNA, labeling miRNA, and/or evaluating a miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, components for in situ hybridization and components for isolating miRNA. Other kits of the invention may include components for making a nucleic acid array comprising miRNA, and thus, may include, for example, a solid support.

### 12. TREATMENTS

A method of treatment is also provided. A subject may be diagnosed with mesothelioma following the administration of medical tests well-known to those in the medical profession. In certain embodiments, the present invention provides methods for the treatment of mesothelioma comprising administering to a subject in need thereof a pharmaceutical composition. Administration of a pharmaceutical composition of the present invention to a subject having mesothelioma may result in one or more clinically desirable outcomes. Such clinically desirable outcomes include reduction of tumor number or reduction of tumor size. Additional clinically desirable outcomes include the extension of overall survival time of the subject, and/or extension of progression-free survival time of the subject. In certain embodiments, administration of a pharmaceutical composition of the invention prevents an increase in tumor size and/or tumor number. In certain embodiments, administration of a pharmaceutical composition of the invention prevents the recurrence of tumors. Administration of a pharmaceutical composition of the present invention results in desirable phenotypic effects. A subject's response to treatment may be evaluated by tests similar to those used to diagnosis the mesothelioma. Response to treatment may also be assessed by measuring biomarkers in blood, for comparison to pre-treatment levels of biomarkers.

The compounds provided herein maybe useful for the treatment of mesothelioma.

Tumor treatments often comprise more than one therapy. As such, in certain embodiments the present invention provides methods for treating mesothelioma comprising administering to a subject in need thereof a pharmaceutical composition of the present invention, and further comprising administering at least one additional therapy.

In certain embodiments, an additional therapy may also be designed to treat mesothelioma. An additional therapy may be a chemotherapeutic agent. An additional therapy may be surgery. An additional therapy may be the use of radiation.

In certain embodiments, an additional therapy may be a pharmaceutical agent that enhances the body's immune system, including low-dose cyclophosphamide, thymostimulin, vitamins and nutritional supplements (e.g., antioxidants, including vitamins A, C, E, beta-carotene, zinc, selenium, glutathione, coenzyme Q-10 and echinacea), and vaccines, e.g., the immunostimulating complex (ISCOM), which comprises a vaccine formulation that combines a multimeric presentation of antigen and an adjuvant.

In certain such embodiments, the additional therapy is selected to treat or ameliorate a side effect of one or more pharmaceutical compositions of the present invention. Such side effects include, without limitation, injection site reactions, liver function test abnormalities, renal function abnormalities, liver toxicity, renal toxicity and central nervous system abnormalities.

In certain embodiments, one or more pharmaceutical compositions of the present invention and one or more other pharmaceutical agents are administered at the same time. In certain embodiments, one or more pharmaceutical compositions of the present invention and one or more other pharmaceutical agents are administered at different times. In certain embodiments, one or more pharmaceutical compositions of the present invention and one or more other pharmaceutical agents are prepared together in a single formulation. In certain embodiments, one or more pharmaceutical compositions of the present invention and one or more other pharmaceutical agents are prepared separately.

In certain embodiments, suitable administration routes of a pharmaceutical composition for the treatment of mesothelioma include, but are not limited to, oral, rectal, transmucosal, intestinal, enteral, topical, suppository, through inhalation, intrathecal, intraventricular, intraperitoneal, intrapleural, intrapericardial, intranasal, intraocular, intratumoral, and parenteral (e.g., intravenous, intramuscular, intramedullary, and subcutaneous). An additional suitable administration route includes chemoembolization. In certain embodiments, pharmaceutical intrathecals are administered to achieve local rather than systemic exposures. For example, pharmaceutical compositions may be injected directly in the area of desired effect (e.g., into a tumor).

In certain embodiments, a pharmaceutical composition of the present invention is administered in the form of a dosage unit (e.g., tablet, capsule, bolus, etc.). In certain embodiments, such pharmaceutical compositions comprise a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, a pharmaceutical composition of the present invention comprises a dose selected from 25 mg, 50 mg, 75 mg, 100 mg, 150 mg, 200 mg, 250 mg, 300 mg, 350 mg, 400 mg, 500 mg, 600 mg, 700 mg, and 800 mg.

In certain embodiments, the compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the formulation.

In certain embodiments, pharmaceutical compositions of the present invention comprise one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulosem and polyvinylpyrrolidone.

In certain embodiments, a pharmaceutical composition of the present invention is prepared using known techniques, including, but not limited to mixing, dissolving, granulating, dragee-making, levigating, emulsifying, encapsulating, entrapping or tabletting processes.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

In certain embodiments, a pharmaceutical composition of the present invention is a solid (e.g., a powder, tablet, and/or capsule). In certain of such embodiments, a solid pharmaceutical composition is prepared using ingredients known in the art, including, but not limited to, starches, sugars, diluents, granulating agents, lubricants, binders, and disintegrating agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present invention is prepared for oral administration. In certain of such embodiments, a pharmaceutical composition is formulated by combining one or more compounds with one or more pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. In certain embodiments, pharmaceutical compositions for oral use are obtained by mixing oligonucleotide and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In certain embodiments, a pharmaceutical composition is prepared for rectal administration, such as a suppositories or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount of the pharmaceutical composition of the present invention is sufficient to prevent, alleviate or ameliorate symptoms of a disease or to prolong the survival of the subject being treated. Determination of a therapeutically effective amount is well within the capability of those skilled in the art.

In certain embodiments, the pharmaceutical composition of the present invention is formulated as a prodrug. In certain embodiments, upon in vivo administration, a prodrug is chemically converted to the biologically, pharmaceutically or therapeutically more active form of the composition. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The following examples are presented in order to more fully illustrate some embodiments of the invention. They should, in no way be construed, however, as limiting the broad scope of the invention.

### EXAMPLES

### Example 1

### Differential Expression of Mesothelioma-Related miRs as Detected by Microarray

### Example 1.I

### Materials and methods

### Example 1.I.a

### Biological Samples

Thirty six Malignant Mesothelioma specimens (from 8 female subjects and 28 male subjects) and 20 matching peritoneum specimens from the time of their resections, were used for miR analysis as described below. There were 10 Stage I/II (Median Survival=26 months) subjects and 26 Stage III/IV subjects (Median Survival=6 months).

### Example 1.I.b.

### Microarray Platform

Custom microarrays were produced by printing DNA oligonucleotide probes representing 903 human microRNAs. Each probe, printed in triplicate, carries up to 22-nt linker at the 3′ end of the microRNA's complement sequence in addition to an amine group used to couple the probes to coated glass slides. 20 μM of each probe were dissolved in 2×SSC+0.0035% SDS and spotted in triplicate on Schott Nexterion® Slide E coated microarray slides using a Genomic Solutions® BioRobotics MicroGrid II according the MicroGrid manufacturer's directions. 22 negative control probes were designed using the sense sequences of different microRNAs. Two groups of positive control probes were designed to hybridize to the microarray (i) synthetic small RNA were spiked to the RNA before labeling to verify the labeling efficiency and (ii) probes for abundant small RNA (e.g. small nuclear RNAs (U43, U49, U24, Z30, U6, U48, U44), 5.8s and 5s ribosomal RNA) are spotted on the array to verify RNA quality. The slides were blocked in a solution containing 50 mM ethanolamine, 1M Tris (019.0) and 0.1% SDS for 20 min at 50° C., then thoroughly rinsed with water and spun dry.

Five μg of total RNA were labeled by ligation (Thomson et al., Nature Methods 2004, 1:47-53) of an RNA-linker, p-rCrU-Cy/dye (Dharmacon), to the 3′-end with Cy3 or Cy5. The labeling reaction contained total RNA, spikes (0.1-20 fmoles), 400 ng RNA-linker-dye, 15% DMSO, 1× ligase buffer and 20 units of T4 RNA ligase (NEB) and proceeded at 4° C. for 1 hr followed by 1 hr at 37° C. The labeled RNA was mixed with 3× hybridization buffer (Ambion), heated to 95° C. for 3 min and then added on top of the microarray. Slides were hybridized 12-16 hr in 42° C., followed by two washes in room temperature with 1×SSC and 0.1% SDS and a final wash with 0.1×SSC.

Arrays were scanned using an Agilent Microarray Scanner Bundle G2565BA (resolution of 10 μm at 100% power). Array images were analyzed using SpotReader software (Niles Scientific).

### Example 1.I.c.

### Data Analysis and Statistics

Kaplan Meier modelling was used to establish the relation between any feature (particularly miR expression and clinical risk factors, such as stage, age, smoking and gender) and prognosis. Cox regression analysis was used to relate between a combination of such features and prognosis. Both KM and Cox regression allow the estimation of how well the time of an event (specifically: death and disease progression) can be predicted for a specific patient with given characteristics, while correcting for ‘censored’ data, i.e. patients for whom no event occurred while within the study, but for whom follow-up was shorter than the time of interest.

The Student t-test was used to determine whether a miR was differentially expressed between two states (for instance, tumor vs. non-tumor, or short vs. long survival), to see if miRs could serve for diagnosis or as predictors of prognosis. As there were many miRs, a multiple hypothesis correction from the standard p<0.05 was made by using the Benjamini-Hochberg method of analysis of “False Discovery Rate” (FDR), which sorts p-values and allows only a false positive rate as pre-set, in this case the rate was set as 0.01 for diagnosis and at 0.1 for prognosis.

### Example 1.II

### Specific miRs are Indicative of Survival in Mesothelioma Subjects

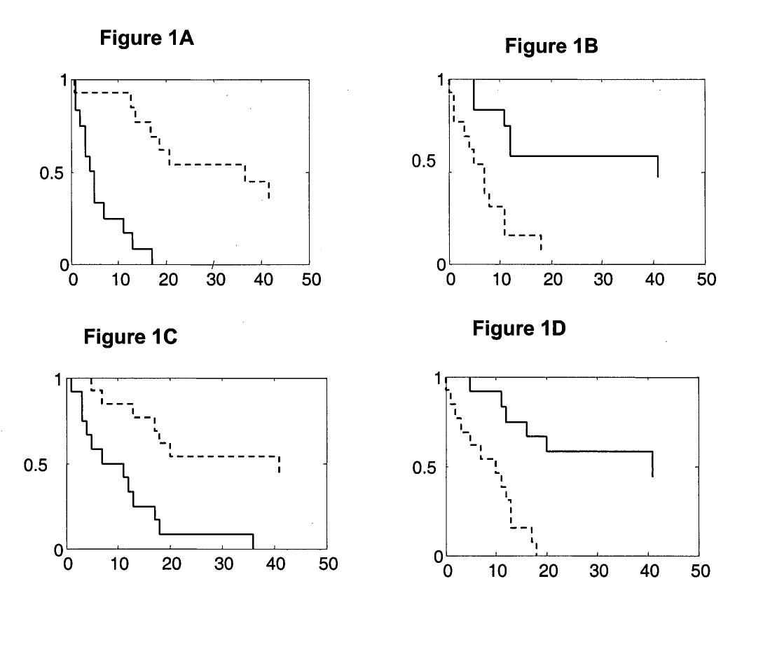

The level of specific miR expression was determined as described in example 1 and indicated in FIG. 1. The p-values according to log rank are as follows: hsa-miR-29c* (SEQ ID NO: 1) p=1.217e-006, hsa-let-7b (SEQ ID NO: 5) p=0.17826, hsa-miR-21* (SEQ ID NO: 11) p=0.22793, hsa-miR-768-5p (SEQ ID NO: 50) p=1.1737e-006. As indicated in FIG. 1, the fraction of survival in patients with higher expression levels of any of hsa-miR-29c* (SEQ ID NO: 1) and miR-768-5p (SEQ ID NO: 50) is higher than in patients with lower expression levels of these miRs. Additionally, the fraction of survival in patients with lower expression levels of any of hsa-let-7b (SEQ ID NO: 5) and hsa-miR-21* (SEQ ID NO: 11) is higher than in patients with higher expression levels of these miRs.

### Example 1.III

### Combination of Expression of Specific miRs are Indicative of Survival in Mesothelioma Subjects

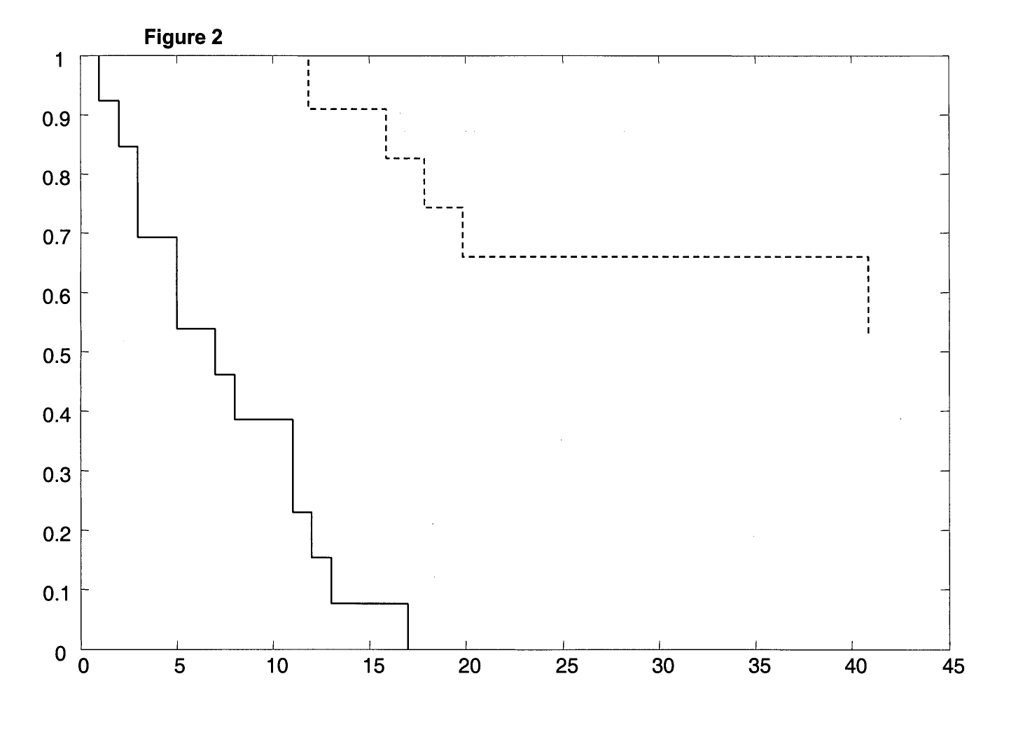

Forward stepwise analysis used Cox regression to form scores based on a linear combination of miRs. MiRs were initially sorted as to their individual p-value. MiRs were successively added to the model and the results of the Cox regression, before and after inclusion were compared. The inclusion criterion for introducing a miR was a p-value of 0.05 and the exclusion criterion was 0.1. As indicated in FIG. 2, the fraction of survival in subjects with lower scores from the combined expression levels of hsa-miR-29c* (SEQ ID NO: 1), hsa-miR-21* (SEQ ID NO: 11) and hsa-miR-34a (SEQ ID NO: 9) is significantly higher than subjects with higher scores. The p-value according to log rank is p=1.5026e-005.

### Example 1.IV

### Combination of Expression of Specific miRs Together with Mesothelioma Stage is Indicative of Survival in Mesothelioma Subjects

Forward stepwise analysis was used to form combinations of miRs as in example 1.III, in tandem with the subject's stage of mesothelioma.

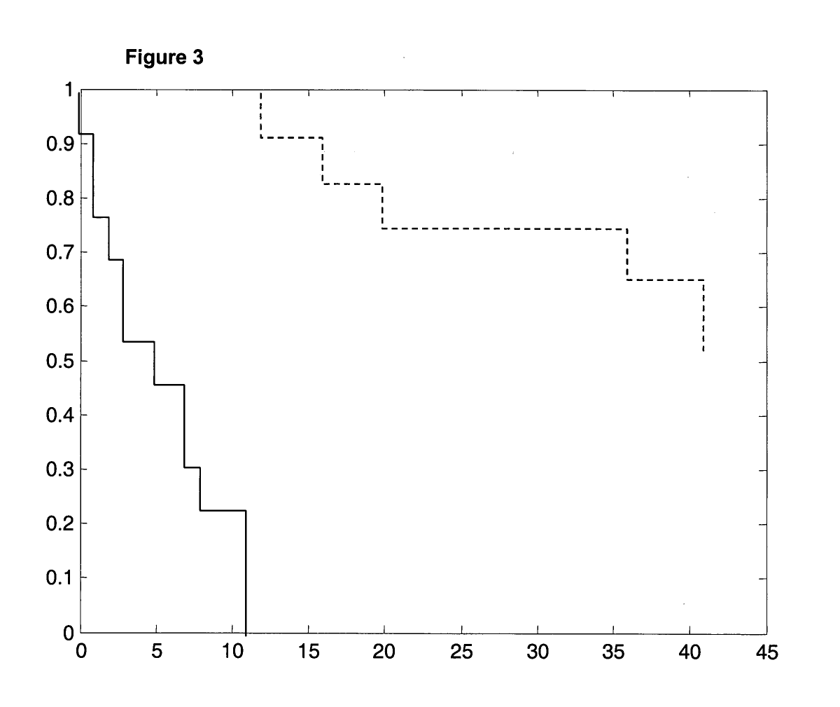

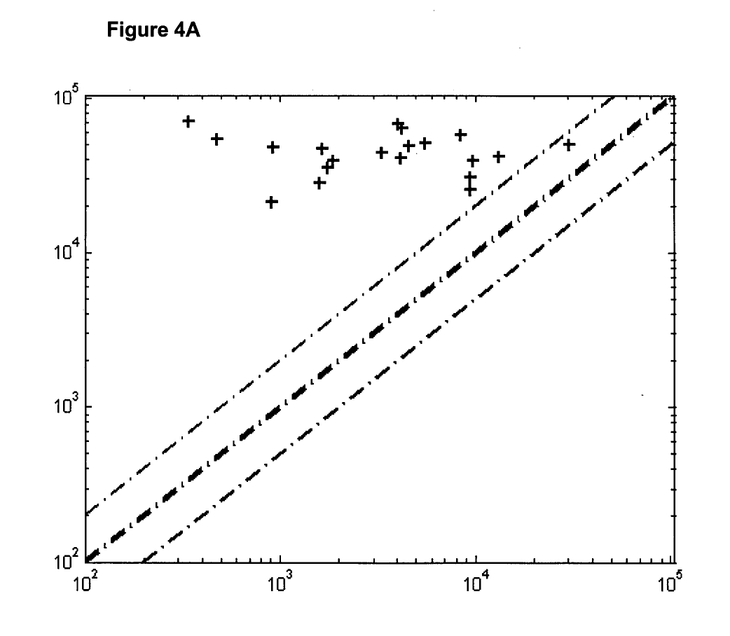

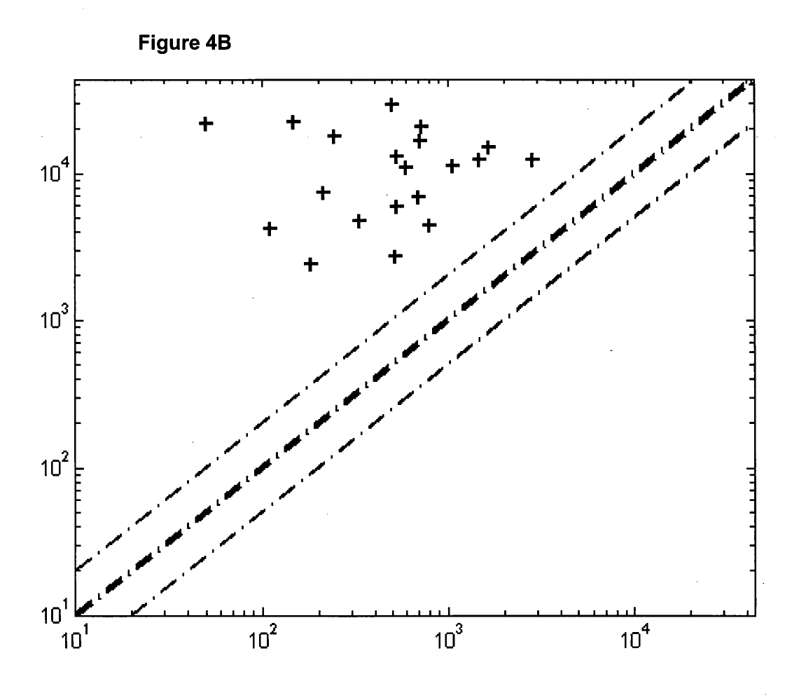

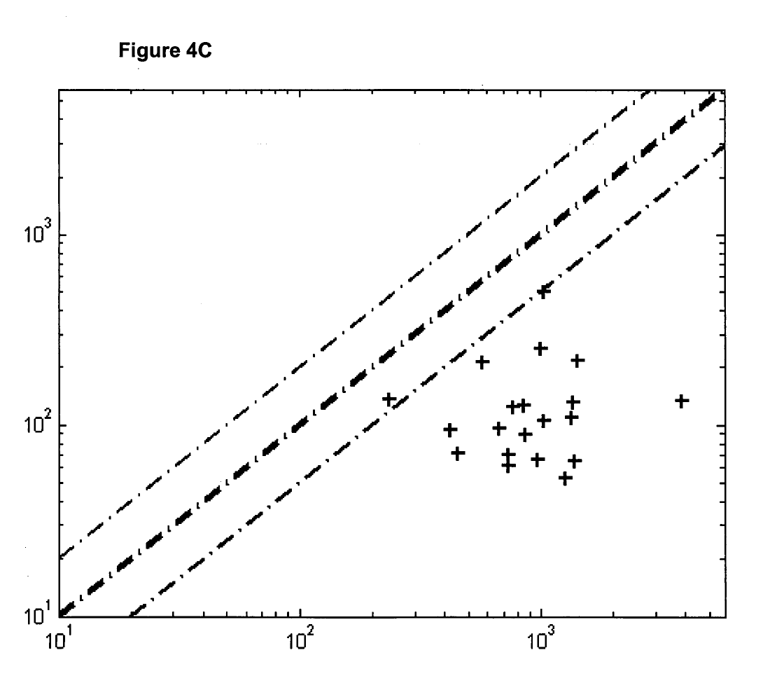

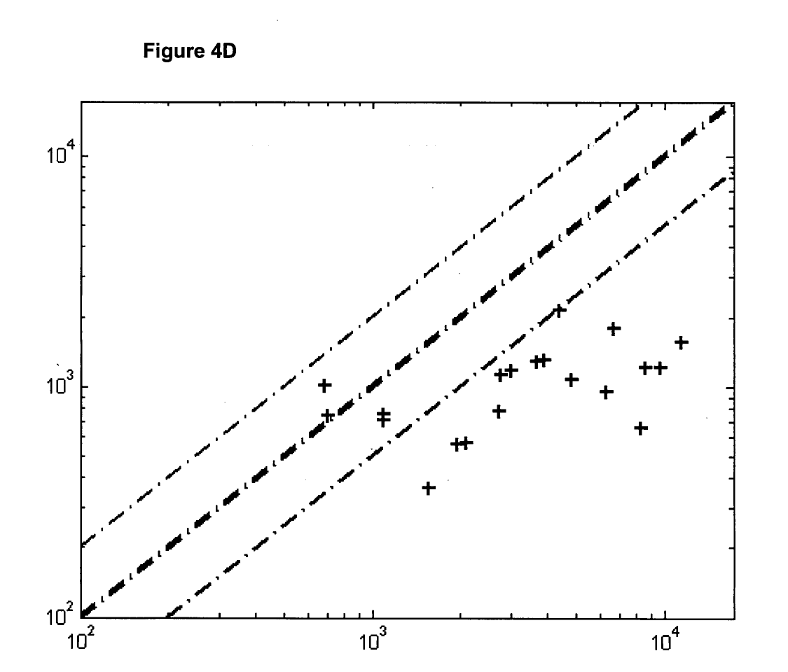

As indicated in FIG. 3, the fraction of survival in subjects with higher scores from the combined expression levels of hsa-miR-29c* (SEQ ID NO: 1), hsa-miR-320 (SEQ ID NO: 3), hsa-miR-21* (SEQ ID NO: 11) and hsa-miR-34a (SEQ ID NO: 9), together with a lower stage of mesothelioma, is significantly higher than in subjects with lower expression scores and a later stage of mesothelioma.

Accordingly, the scores from the combination of expression levels of these miRs together with stage of the mesothelioma, is a significantly good predictor of the survival of mesothelioma patients after surgical operation. The p-value according to log rank is p=9.0052e-006.

### Example 1.V

### Normal Abdominal Mesothelium Vs. Mesothelioma in Lung Pleura

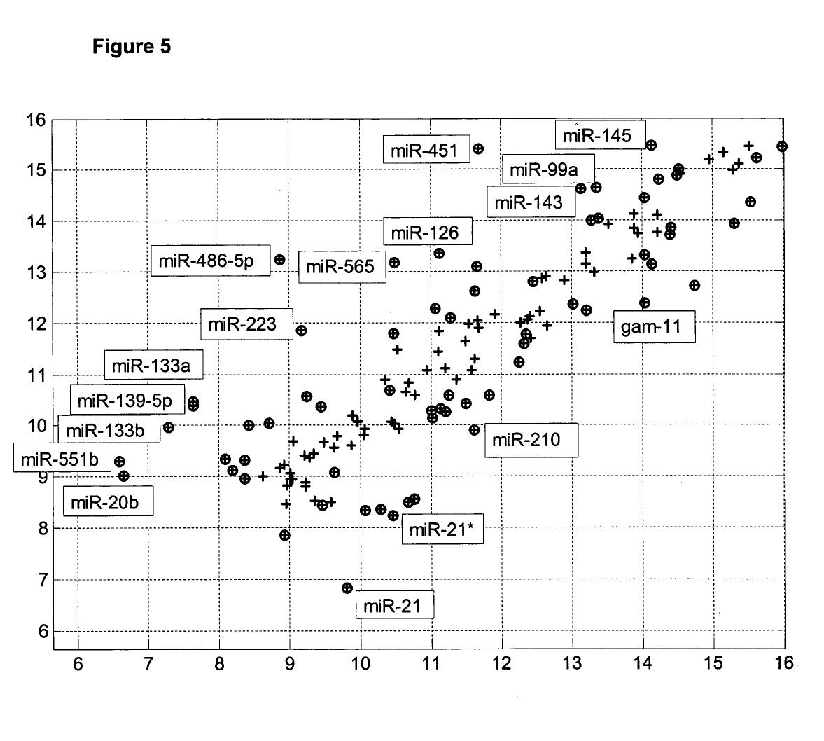

Paired t-test was used for 20 patients having a sample from mesothelioma tumor as well as a normal sample from the abdominal mesothelium. 136 miRs were expressed in at least one set of samples (tumor/normal) and were tested for differential expression. False Discovery Rate (FDR) of 0.01 was used in order to deal with multiple hypotheses. The results included 66 miRs that were significantly differentially expressed between mesothelioma tumors and normal abdominal mesothelium, for example, hsa-miR-133a (SEQ ID NO: 23), hsa-miR-20b (SEQ ID NO: 32), hsa-miR-551b (SEQ ID NO: 30), hsa-miR-133b (SEQ ID NO: 28), hsa-miR-139-5p (SEQ ID NO: 26), hsa-miR-223 (SEQ ID NO: 21), hsa-mir-486-5p (SEQ ID NO: 17), hsa-miR-565 (SEQ ID NO: 19), hsa-miR-126 (SEQ ID NO: 15), hsa-miR-451 (SEQ ID NO: 13), hsa-miR-99a (SEQ ID NO: 38), hsa-miR-145 (SEQ ID NO: 36), hsa-miR-143 (SEQ ID NO: 51), hsa-miR-21* (SEQ ID NO: 11), hsa-miR-21 (SEQ ID NO: 34), hsa-miR-210 (SEQ ID NO: 40) and gam11 (SEQ ID NO: 46) noted in FIG. 5.

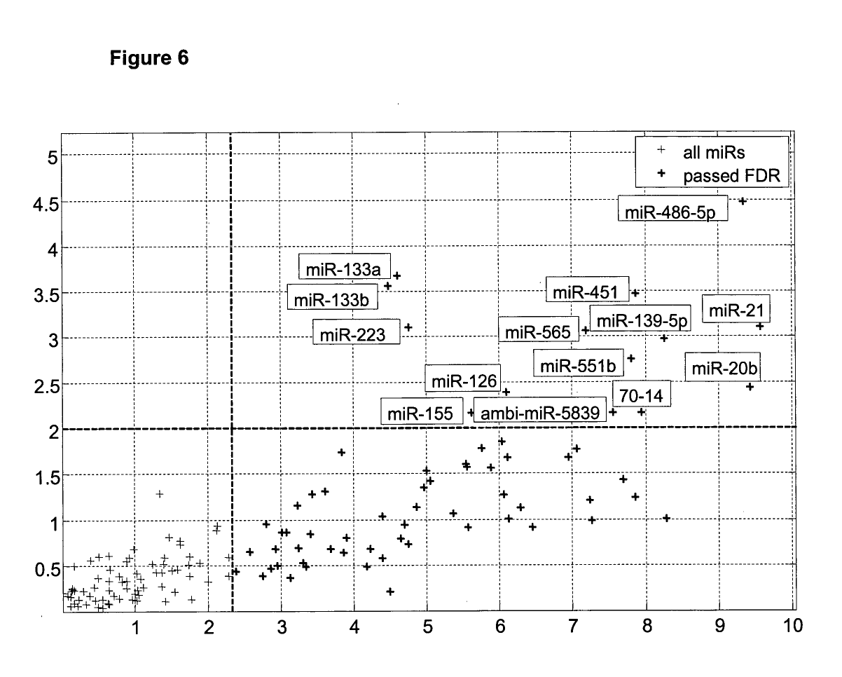

FIG. 6 is a reduced volcano plot for the difference between expression in tumor and normal samples. The X-axis represents −log₁₀ (p-value) of displayed miRs and Y-axis the absolute fold change in units of log₂. The top-right part of figure depicts the following miRs with high fold change and high significance: hsa-miR-133a (SEQ ID NO: 23), hsa-miR-133b (SEQ ID NO: 28), hsa-miR-223 (SEQ ID NO: 21), hsa-miR-126 (SEQ ID NO: 15), hsa-miR-155 (SEQ ID NO: 42), ambi_miR_{—}5893 (SEQ ID NO: 48), 70_{—}14 (SEQ ID NO: 44), hsa-miR-551b (SEQ ID NO: 30), hsa-miR-565 (SEQ ID NO: 19), hsa-miR-451 (SEQ ID NO: 13), miR-139-5p (SEQ ID NO: 26), hsa-miR-20b (SEQ ID NO: 32), hsa-miR-21 (SEQ ID NO: 34) and hsa-mir-486-5p (SEQ ID NO: 17). These miRs are therefore highly significant and potentially useful.

TABLE 1
Specific miRs are indicative of survival, prognosis
and/or diagnostic of mesothelioma subjects

| | miR | Hairpin |
|---|---|---|
| microRNA | SEQ ID | SEQ ID |
| name¹ | NO: | NO: |
| hsa-miR-29c* | 1 | 2 |
| hsa-miR-320 | 3 | 4 |
| hsa-let-7b | 5 | 6 |
| hsa-miR-768-3p | 7 | 8 |
| hsa-miR-34a | 9 | 10 |
| hsa-miR-21* | 11 | 12 |
| hsa-miR-451 | 13 | 14 |
| hsa-miR-126 | 15 | 16 |
| hsa-mir-486-5p | 17 | 18 |
| hsa-miR-565 | 19 | 20 |
| hsa-miR-223 | 21 | 22 |
| hsa-miR-133a | 23 | 24, 25 |
| hsa-miR-139-5p | 26 | 27 |
| hsa-miR-133b | 28 | 29 |
| hsa-miR-551b | 30 | 31 |
| hsa-miR-20b | 32 | 33 |
| hsa-miR-21 | 34 | 35 |
| hsa-miR-145 | 36 | 37 |
| hsa-miR-99a | 38 | 39 |
| hsa-miR-210 | 40 | 41 |
| hsa-miR-155 | 42 | 43 |
| 70_14 | 44 | 45 |
| gam11 | 46 | 47 |
| ambi_miR_5893 | 48 | 49 |
| hsa-miR-768-5p | 50 | 8 |
| hsa-miR-143 | 51 | 52 |

¹Sequences 70_14, gam11 and ambi_miR_5893 were cloned at Rosetta Genomics. For all the other sequences the microRNA name is the miRBase registry name (release 10).

### Example 2

### Time-to-Progression and Survival Related Expression of hsa-miR-29c* (SEQ ID NO: 1) in Training and Test Sets, as Detected in Microarray and Validated by PCR.

### Example 2.I

### Materials and Methods

### Example 2.I.a

### Biological Samples

One hundred and forty two mesothelioma tumors specimens were obtained under the auspices of IRB approved tissue procurement protocols either at the National Cancer Institute, Bethesda Md. (1989-1996), or at the Karmanos Cancer Institute (KCI), Detroit Mich. (1997-2005). Specimens were obtained fresh from the operating room during cytoreductive surgery or biopsy for diagnosis and immediately snap frozen in liquid nitrogen and stored at −80° C. Slides were available for pathologic re-review (MC) of the snap frozen specimens for all but 11 specimens. All specimens had immunohistochemical evidence of mesothelioma with positive staining for WT1, cytokeratins, calretinin, and the absence of staining for CEA and B72.3. Demographics including age, sex, stage, mesothelioma histology, time to progression from surgery, time to death from surgery, were recorded and current as of September 2008.

A training set of 37 specimens and a test set of 92 specimens were used. The training set specimens exclusively originated from KCI, while the test set was a mixture of specimens from KCI and NCI.

As can be seen in table 2, the training set and the test set were similar with regard to sex, age, histology, stage, and treatment options with the majority having cytoreductive surgery.

TABLE 2
Patient Demographics

| | Training Set (n = 37) | Test Set (n = 92) |
|---|---|---|
| Age | 63 + 1 (43-78) yrs. | 62 + 1 (34-87) yrs. |
| Sex | 9 Female, 28 Male (76%) | 16 Female, 76 Male (83%) |
| Stage | I/II: 10 (37%); | I/II: 29 (46%); |
| | III/IV: 27 | III/IV: 63 |
| Histology | 23 Epithelial (62%); | 55 Epithelial (60%); |
| | 14 Biphasic or Sarcomatoid | 37 Biphasic or Sarcomatoid |
| Cytoreductive | 34 Y(92%); 3 N | 83 Y (90%); 9 N |
| Surgery | | |

### Example 2.I.b

### Microarray Platform

Total RNA was extracted from both the training and the test sets using the mirVANA microRNA isolation kit (Ambion). ˜900 DNA oligonucleotide probes representing microRNAs were spotted in triplicate on coated microarray slides (Nexterion® Slide E, Schott, Mainz, Germany).

3-5 μg of total RNA were labeled by ligation of an RNA-linker, p-rCrU-Cy/dye (Dharmacon, Lafayette, Colo.; Cy3 or Cy5) to the 3′ end. Slides were incubated with the labeled RNA for 12-16 hr at 42° C. and then washed twice. Arrays were scanned at a resolution of 10 μm, and images were analyzed using SpotReader software (Niles Scientific, Portola Valley, Calif.). Microarray spots were combined and signals normalized.

### Example 2.I.c

### Data Analysis and Statistics

Patients were grouped by their retrospective prognosis. Specifically, good prognosis was defined as a time to progression (TTP) greater than or equal to 12 months for those patients in whom progression was documented by radiographic or histologic evidence of progression of disease. Dichotomous differentiation into good or bad survival was by survival from the time of surgery greater or equal to 15 months.

Demographic (gender, age etc.) and clinical features (histological type, stage, lymph node involvement and form of chemotherapy) were tested as to their prediction of good or poor prognosis. Analysis was by Kaplan Meier survival analysis and evaluation by log-rank. A p-value less than 0.05 were taken to be significant.

Only microRNAs which had a median signal higher than signal background levels (normalized fluorescence signal of ˜300) in at least one of the two groups were tested. Expression levels were log-transformed (base 2) and then normalized by fitting to a second-degree polynomial. Thereafter, all calculations were performed in normalized log-space. Expression levels between groups of samples were compared using the Mann-Whitney non-parametric test. Corrections for multiple comparisons were performed using the Benjamini-Hochberg False Discovery Rate (FDR) method. Survival time course was studied using the Kaplan Meier method, and groups were compared using log rank test. For the training set the separation was based on the median value of microRNA expression. This value was then taken as the threshold for the splitting of the test set into two groups. An additional correction for multiple hypothesis testing was performed by 100 random re-allocations of the microRNA patterns to patients, and comparison of the p-value for the true best microRNA survival prediction with that obtained by the randomly generated patterns. Multivariate analysis of miR expression and of demographic and clinical features was performed using Cox regression.

### Example 2.I.d

### qRT-PCR

12 microRNAs were selected for quantitative real-time PCR (qRT-PCR) analysis. Seven of these microRNAs: hsa-miR-29c* (SEQ ID NO: 1), hsa-miR-210 (SEQ ID NO: 40), hsa-miR-199b-5p (SEQ ID NO: 53), hsa-miR-451 (SEQ ID NO: 13), hsa-miR-99a (SEQ ID NO: 38), hsa-miR-221 (SEQ ID NO:54), hsa-miR-150 (SEQ ID NO: 55) were selected as differential probes for prognosis and five non-differential microRNAs (hsa-miR-181a (SEQ ID NO: 56), hsa-let-7c (SEQ ID NO: 57), hsa-miR-193a-5p (SEQ ID NO: 58), hsa-miR-27b (SEQ ID NO: 59), hsa-miR-339-5p (SEQ ID NO: 60)) were chosen for signal normalization. Sixteen samples, eight with good prognosis and eight with poor prognosis were selected for analysis. MicroRNA amounts were quantified using a qRT-PCR method as follows: RNA was incubated in the presence of poly (A) polymerase (PAP; Takara-2180A), MnCl₂, and ATP for 1 hour at 37° C. Then, using an oligodT primer harboring a consensus sequence, reverse transcription was performed on total RNA using SuperScript II RT (Invitrogen, Carlsbad, Calif.). Next, the cDNA was amplified by RT-PCR; this reaction contained a microRNA-specific forward primer, a TaqMan probe complementary to the 3′ of the specific microRNA sequence as well as to part of the polyA adaptor sequence, and a universal reverse primer complementary to the consensus 3′ sequence of the oligodT tail.

TABLE 3
Sequences used in PCR validation:

| | SEQ | Hairpin | Specific | SEQ | | SEQ |
|---|---|---|---|---|---|---|
| micro | ID | SEQ ID | Forward | ID | Mgb | ID |
| RNA | NO: | NO: | sequences | NO | sequence | NO |
| hsa- | 40 | 41 | CAGTCATTTGG | 70 | CCGTTTTTTT | 82 |
| miR- | | | GTGAGGTAGTA | | TTTTTAACCA | |
| 210 | | | GGTTGT | | TAC | |
| hsa- | 53 | 61 | CAGTCATTTGG | 71 | CCGTTTTTTT | 83 |
| miR- | | | GAAACCGTTAC | | TTTTTAACTC | |
| 199b- | | | CATTAC | | AGT | |
| 5p | | | | | | |
| hsa- | 1 | 2 | CAGTCATTTGG | 72 | CCGTTTTTTT | 84 |
| miR- | | | GAACATTCAAC | | TTTTTACTCA | |
| 29c* | | | GCTGTC | | CCG | |
| hsa- | 13 | 14 | CAGTCATTTGG | 73 | CCGTTTTTTT | 85 |
| miR- | | | GAACCCGTAGA | | TTTTTCACAA | |
| 451 | | | TCCGAT | | GAT | |
| hsa- | 38 | 39 | CAGTCATTTGGC | 74 | CCGTTTTTTT | 86 |
| miR- | | | TCTCCCAACCCT | | TTTTTCACTG | |
| 99a | | | TGTA | | GTA | |
| hsa- | 54 | 62 | CAGTCATTTGGC | 75 | CCGTTTTTTT | 87 |
| miR- | | | TCCCTGTCCTCC | | TTTTTCGTGA | |
| 221 | | | AGGA | | GCT | |
| hsa- | 55 | 63 | CAGTCATTTGGC | 76 | CCGTTTTTTT | 88 |
| miR- | | | TGACCGATTTCT | | TTTTTGAAC | |
| 150 | | | CCTG | | ACCA | |
| hsa- | 56 | 64, 65 | CAGTCATTTGGC | 77 | CCGTTTTTTT | 89 |
| miR- | | | CCCAGTGTTTAG | | TTTTTGAAC | |
| 181a | | | ACTA | | AGAT | |
| hsa- | 57 | 66 | CAGTCATTTGG | 78 | CGTTTTTTTT | 90 |
| let-7c | | | GCTGTGCGTGT | | TTTTCAGCC | |
| | | | GACAGC | | GCT | |
| hsa- | 58 | 67 | CAGTCATTTGGC | 79 | CGTTTTTTTT | 91 |
| miR- | | | TGGGTCTTTGCG | | TTTTCATCTC | |
| 193a- | | | GGCG | | GC | |
| 5p | | | | | | |
| hsa- | 59 | 68 | CAGTCATTTGG | 80 | CGTTTTTTTT | 92 |
| miR- | | | GAGCTACATTG | | TTTTGAAAC | |
| 27b | | | TCTGCT | | CCA | |
| hsa- | 60 | 69 | CAGTCATTTGG | 81 | CGTTTTTTTT | 93 |
| miR- | | | GTTCACAGTGG | | TTTTGCAGA | |
| 339- | | | CTAAGT | | ACT | |
| 5p | | | | | | |

| SEQ ID NO: 94 | |
|---|---|
| Reverse sequence | GCGAGCACAGAATTAATACGAC |

The cycle threshold (Ct, the PCR cycle at which probe signal reaches the threshold) was determined for each microRNA. To allow comparison with results from the microarray, each value received was subtracted from 50. This 50-Ct (50_{Ct}) expression for each microRNA for each patient was compared with the signal obtained by the microarray method. Linear regression for the microRNA readings over all patients was used to model 50_{Ct} by microarray values. Using this model the threshold for the separation between high and low expression samples was transferred from microarray to qRT-PCR readings and used for Kaplan-Meier analysis.

### Example 2.II

### Training Set: Time-to-Progression and Survival Related Expression of hsa-miR-29c* (SEQ ID NO: 1), and in Relation to Stage of Mesothelioma.

### Example 2.II.a

### Training Set—Time to Progression

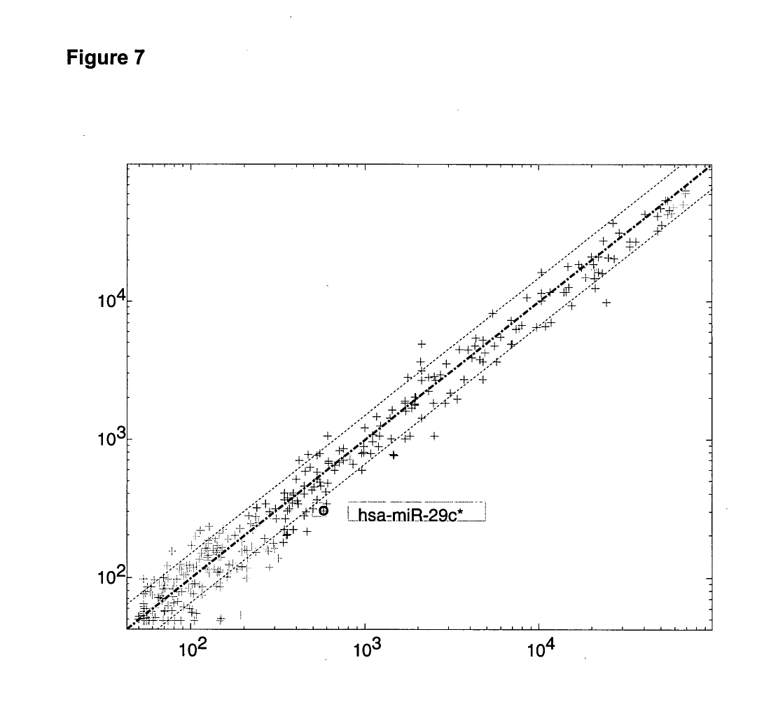

The median time to progression for the individuals in the training set was 8 months. As indicated in FIG. 7, in the comparison of good prognosis patients (TTP>12 months, n=10) with patients with TTP<12 months (n=24) and with three censored patients omitted, hsa-miR-29c* (SEQ ID NO: 1) had significantly different expression (p value 0.000355, fold change 1.8) after adjusting for the FDR.

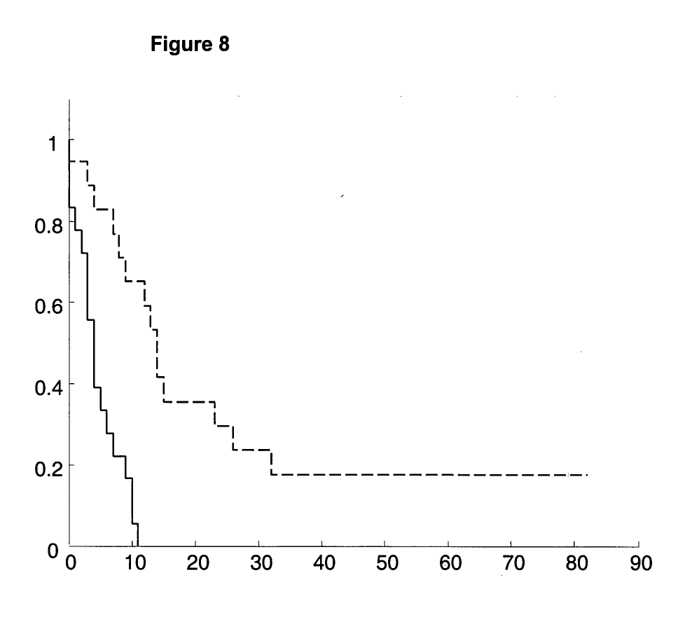

As seen in the Kaplan Meier plot of FIG. 8, when the median expression of hsa-miR-29c* (SEQ ID NO: 1) 8.8 was used as a cutoff, two groups with significantly different TTP were seen (4 months vs. 14 months, p=0.002). An elevated level of hsa-miR-29c* (SEQ ID NO: 1) was associated with an increased time to progression.

The significance of this result was tested by random re-labeling of the samples: microRNA profiles were randomly reallocated to patients 100 times; for each random set, each microRNA was used to divide patients into two groups with signals higher or lower than its median. Kaplan Meier analysis and the log rank test were then used to obtain a p-value for the significance of the association of each microRNA with a difference in progression-free survival. This procedure gave a distribution of “best p-values” for a random profile, while reflecting the specific population. The 100 reallocations did not result in a single case for which the most significant microRNA was more significant (lower p-value) than the real value obtained for hsa-miR-29c* in the training set data. This reflects a secondary adjusted p-value p<0.01 for a situation of multiple hypothesis testing.

In the training set, patients in stage I/II MM had a significantly longer time to progression (19.5 months) than those with stage III/IV (7 months, p=0.005) validating previous data regarding the influence of stage on time to progression. In a multivariate analysis for the training set, stage was the only independent predictor of time to progression (Hazard Ratio 3.8, 0.0099, Confidence Interval: 1.3-10.2).

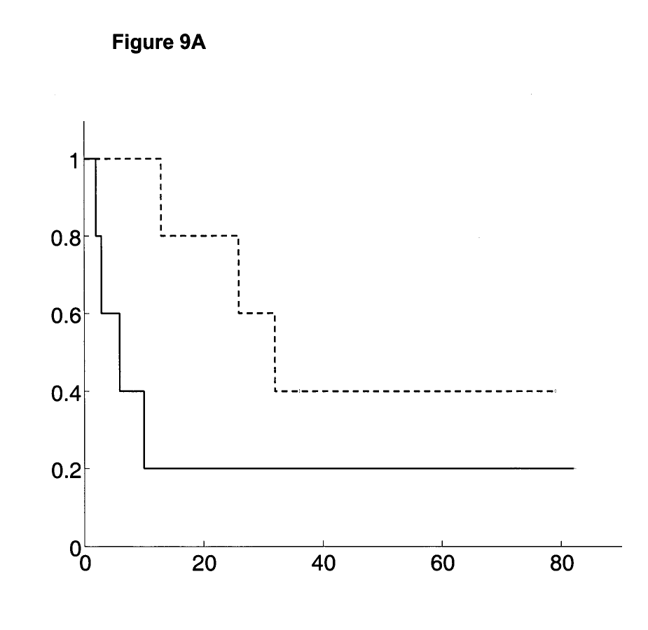

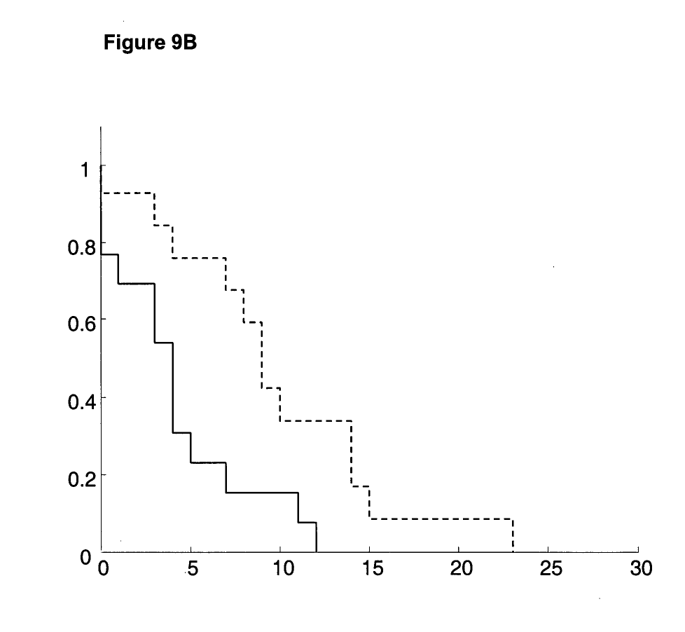

The expression of hsa-miR-29c* (SEQ ID NO: 1) did not differ between samples from patients stage I/II and stage III/IV. Within each of the groups, the expression levels of hsa-miR-29c* (SEQ ID NO: 1) maintained an association with progression-free survival, which was significant for samples of stages III/IV (p=0.02, FIG. 9B) but did not reach statistical significance for Stages I/II, due to the small number of samples in this subgroup (n=10, p=0.15, FIG. 9A).

### Example 2.II.b

### Training Set—Survival

The median survival for the training set was 12 months. Six patients of the training set survived at least 15 months, while 28 died of the disease within this period and 3 patients, who were censored, died from other causes within 15 months. Of the leading microRNAs differentiating survivors from non-survivors, hsa-miR-29c* (SEQ ID NO: 1) was the best discriminator (p=0.006, fold change 1.7).

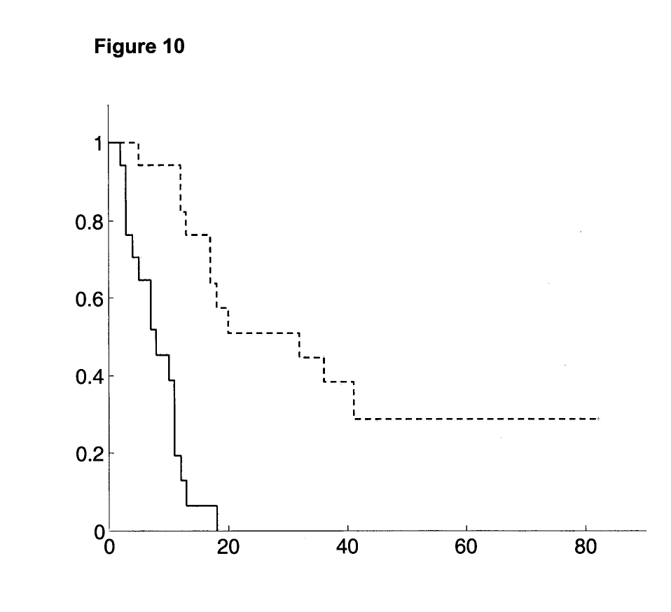

When the median expression of hsa-miR-29c* (SEQ ID NO: 1), 8.8, was used as the cut-off point, two distinct groups of mesothelioma patients with significantly different survival curves were seen (median survival of 8 months vs. 32 months, p=0.000019), as indicated in FIG. 10.

### Example 2.III

### Test Set: Time-to-Progression and Survival Related Expression of Hsa-miR-29c* (SEQ ID NO: 1), and in Relation to Stage of Mesothelioma

### Example 2.III.a

### Test Set—Time to Progression

The median time to progression for the 92 individuals of the test set was 9 months.

In a multivariate analysis for the test set, stage was the only independent demographic or clinical predictor of time to progression (p=0.000012, Hazard Ratio 3.8, Confidence Interval (CI): 2.1-7.1).

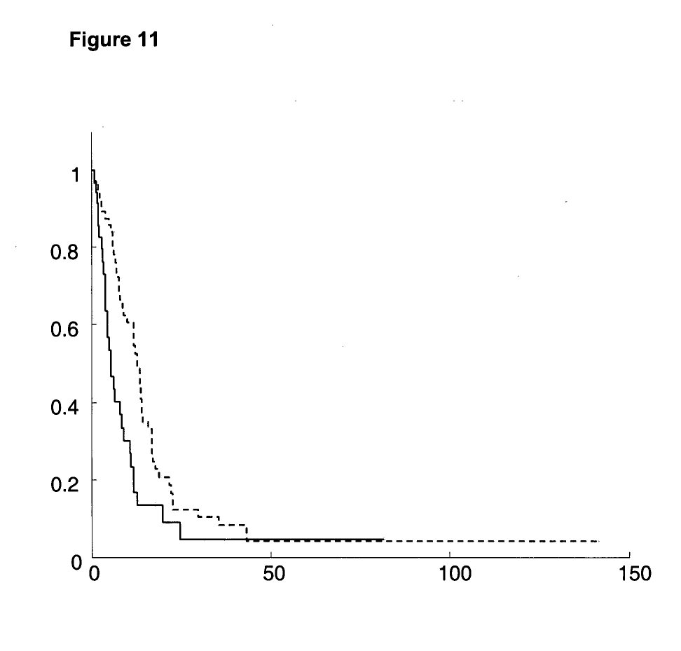

As seen in FIG. 11, when median expression of hsa-miR-29c* (SEQ ID NO: 1) (8.8) from the training set was used as a cutoff, two groups in the test set with significantly different TTP were seen (5.5 months vs. 12.8 months, p=0.008).

### Example 2.III.b

### Test Set—Survival

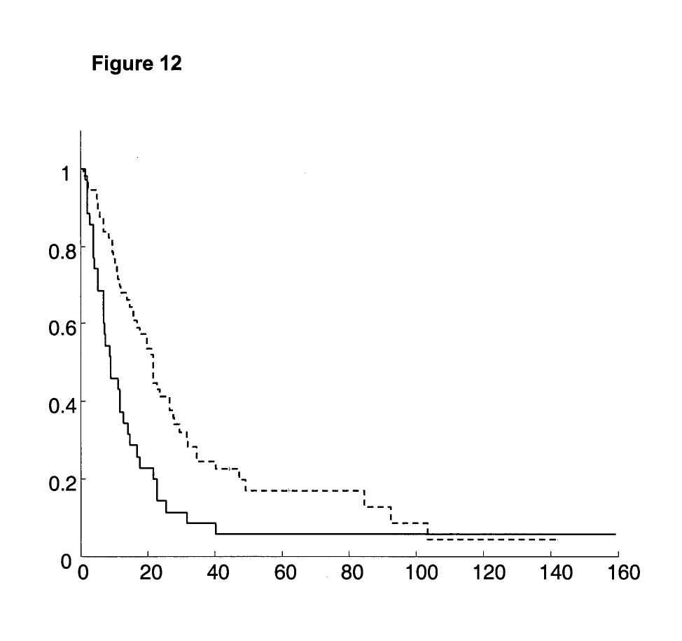

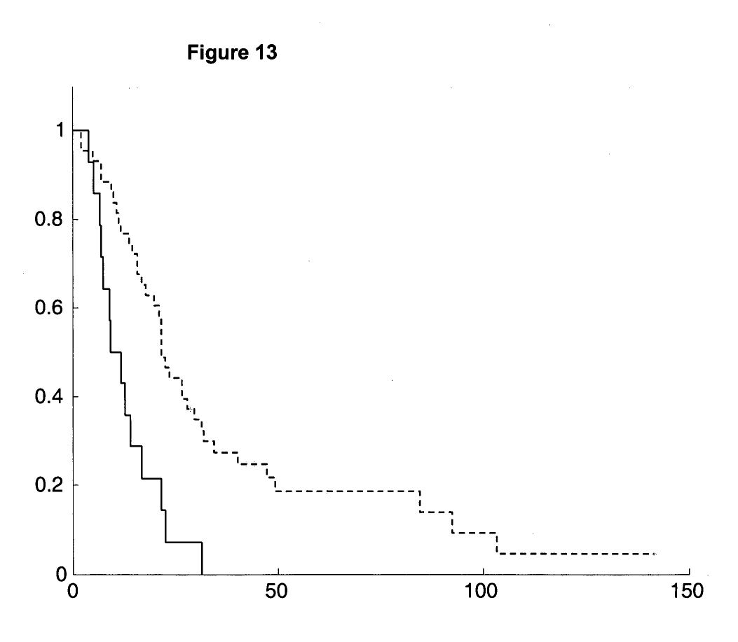

The median overall survival for the test set was 16 months. hsa-miR-29c* upregulation was associated with good prognosis (survival >15 months, p=0.0014, hazard ratio 1.83). When differential expression of hsa-miR-29c* (SEQ ID NO: 1) was investigated in the test set, the microRNA's expression was highly prognostic. The threshold of expression determined in the training set (8.8) separated the patients in the test set into two groups with median survival times of 9.1 months vs. 21.6 months (p=0.0026, FIG. 12). hsa-miR-29c* (SEQ ID NO: 1) could also separate epithelial mesotheliomas into a long survival group (median survival, 21.6 months) and a short survival group (median survival, 9.1 months, p=0.0005) as indicated in FIG. 13.

In a multivariate analysis for the test set, stage was the only independent demographic or clinical predictor of survival (p=0.00016, Hazard Ratio 3.1, Confidence Interval 1.7-5.5).

### Example 2.IV

### RT-PCR Validation of hsa-miR-29c* (SEQ ID NO: 1) Expression

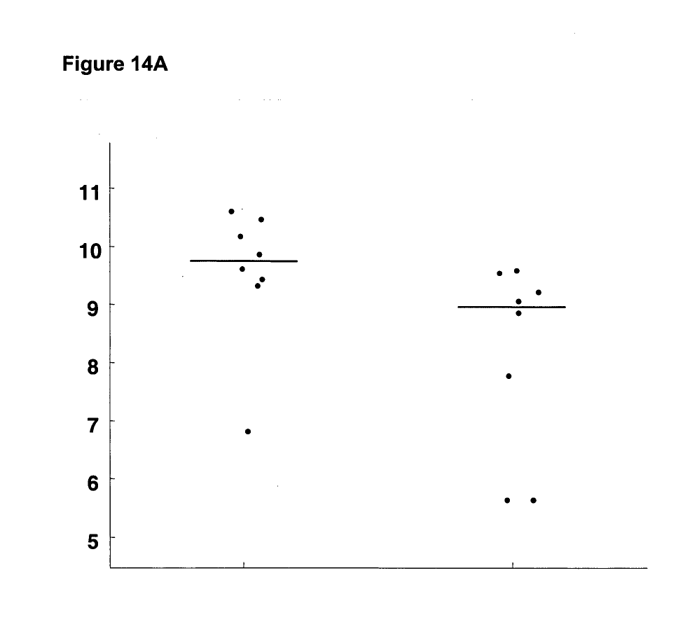

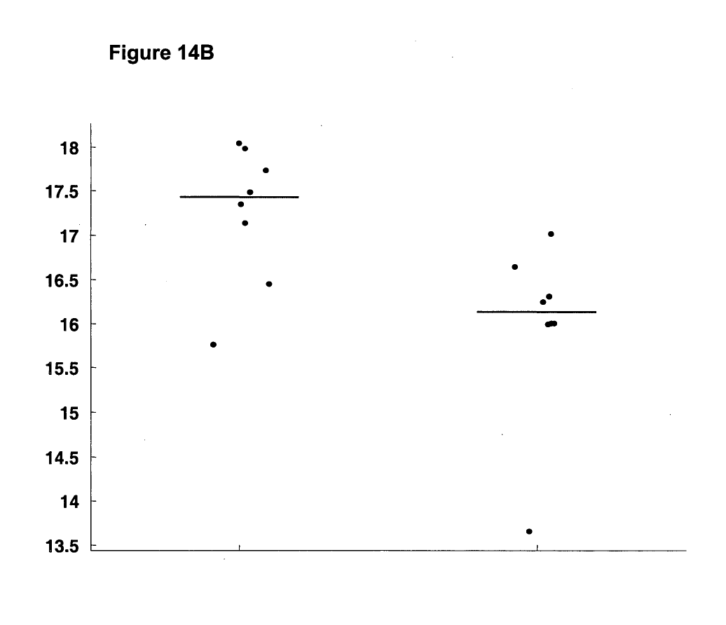

As noted in examples 2.II and 2.III above, hsa-miR-29c* (SEQ ID NO: 1) was found to be significant in both the training set and the test set. Validation of these results was performed using quantitative RT-PCR. RNA was isolated from 16 test set patients, 8 of whom had survival of more than 15 months and 8 with survival equal to or less than 15 months. The microRNA expression measurement by the custom microRNA array, and was compared to the expression levels measured by qRT-PCR, as described in example 2.I.d above. As is seen in FIG. 14, hsa-miR-29c* (SEQ ID NO: 1) was significantly higher in the good prognosis group for both the custom array and the PCR. Cₜ, the raw signal cycle threshold, was used as the measure of expression.

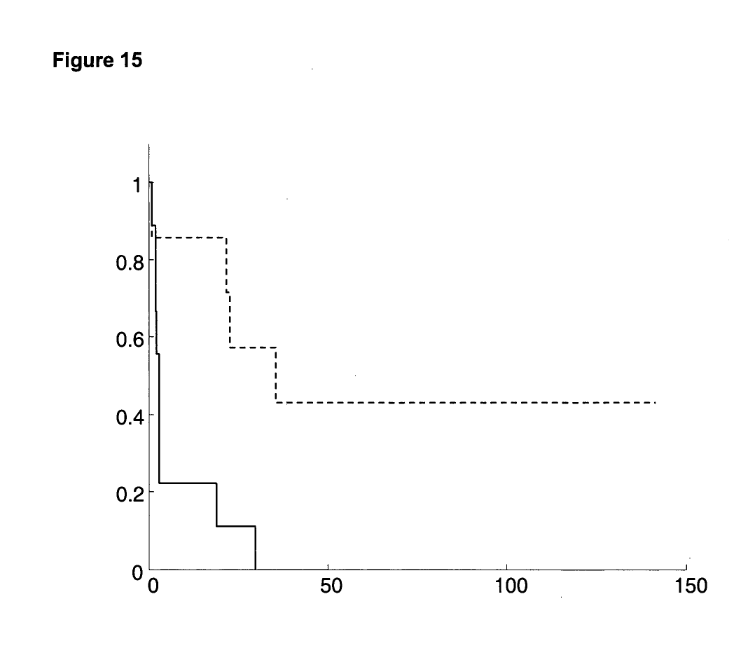

The correlation coefficient between microarray expression and the PCR was 0.649, and using the mathematical regression line, the median expression of 9.4 for the array correlated with a 50-Cₜ measurement of 16.8. Using these cut-off values, Kaplan Meier curves were reconstructed for the 16 individuals, and as is seen in FIG. 15, this cutoff using the qRT-PCR data was able to divide the mesotheliomas into a good and bad prognosis group (median survivals, 2.8 months vs. 35.4 months, p=0.015).

### Example 3

### In vitro functional analyses of hsa-miR29c*

### Example 3.I

### Materials and Methods

### Example 3.I.a

### Cell Lines, miRNA Oligonucleotides and Transfection

Mesothelioma cell lines H2595, HP-1, and Hmeso were grown in 1×DMEM supplemented with sodium pyruvate and high glucose and 10% fetal bovine serum (FBS) (Invitrogen, Carlsbad, Calif.).

Pre-miR™ miRNA mimic (miR-29c*) and negative control were purchased from Ambion (Austin, Tex.). For transfection of miR-29c* (SEQ ID NO: 1) into H2595, HP-1 and Hmeso, cells were plated at 60-70% confluency in 10 cm dishes 24 hours prior to transfection. Two hours before transfections, the medium was changed to antibiotic-free DMEM/10% FBS. A total of 40 nM of miRIDIAN miR-29c* (SEQ ID NO: 1) mimic or negative control was complexed with 60 ul of Lipofectamine 2000 as recommended by the manufacturer (Invitrogen, Carlsbad, Calif.). Transfections were removed after 4 hours incubation and replaced with fresh DMEM/10% FBS medium. 48 hours upon post transfection, cells were trypsinized, counted and assayed for proliferation, colony formation on soft agar, wound closure, and matrigel invasion in triplicate experiments.

### Example 3.I.b

### RT-PCR Validation of miR-29c* (SEQ ID NO: 1) Delivery

Stem-loop technology in its end-point PCR modification (Varkonyi-Gasic et al., Plant Methods, 3: 12, 2007) was used to validate miR-29c* (SEQ ID NO: 1) delivery to the cells. Briefly, a stem-loop RT primer was designed to anneal to the 3′-end of miR-29c* (SEQ ID NO: 1) and ensure extension of the product size during the PCR stage. In the PCR stage, a miR-29c*-specific forward primer was used, and a stem-loop-specific oligonucleotide as a reverse primer, as detailed in Table 3 below.

TABLE 4
Sequences used in the Stem-loop RT-PCR vali-
dation of miR-29c* (SEQ ID NO: 1) delivery

| | | SEQ |
|---|---|---|
| | | ID |
| RT-PCR SEQUENCE | | NO: |
| stem-loop | 5′GTCGTATCCAGTGCAGGGTCCGAGGTATTCGCA | 95 |
| RT primer | CTGGATACGACGAACACCAGG-3′ | |
| specific | 5′-TGACCGATTTCTCCTGG-3′ | 96 |
| forward | | |
| primer | | |
| stem-loop- | 5′-GTGCAGGGTCCGAGGT-3′ | 97 |
| specific | | |
| oligo- | | |
| nucleotide | | |
| (reverse | | |
| primer) | | |

RT-PCR was performed on total miR-containing RNA isolated from cultured MM cells using pulsed RT and PCR protocols. The PCR product was visualized by ethidium bromide staining in 4% MetaPhor agarose (Rockland, Me.).

### Example 3.I.c.

### Cell Proliferation Assay

Three thousand transfected cells in 100 μl DMEM/10% FBS were plated in flat bottom 96-well plates in six replicates and incubated at 37° C. for 48 hours. 20 μl of CellTiter-Blue reagent (Promega # TB317, USA) was added to each well and incubated for another 1-3 hours at 37° C. Optical density (OD) values were measured at 560/590 nm using the universal plate reader (Universal Reader Victor³; PerkinElmer Life and Analytical Sciences, USA).

### Example 3.I.d

### Scratch Assay (Wound Closure Assay)

One day before the experiment, 2×10⁵ cells/well were seeded into 6-well plates. Two to three vertical scratches per well were made using a 1 mL pipette tip. The cells were then washed with 1×PBS and the medium changed in order to avoid conditioning of the media with floating cells. Scratched areas were marked with a fine pointer and the scratch width (6 measurements per well) was measured immediately after the scratch (zero hour point) and 24 hours later. The percent of the scratch closure, reflecting the motility rate, was calculated.

### Example 3.I.e

### Matrigel Cell Invasion Assay

Cell invasion assay was performed using the BD Biocoat Matrigel Invasion Chambers according to the manufacturer's protocol. One million cells were seeded onto the inserts (8 uM pore sized polycarbonate membrane) coated with a thin layer of Matrigel Basement Membrane Matrix w/o phenol red (BD Biosciences) diluted at 1:100 in PBS. The plates were incubated for 48 hours at 37° C. The cells that invaded through the basement matrigel to the lower surface of the membrane were fixed and stained with Giemsa solution, and counted under the microscope. At least 10 different fields were counted and photographed. All the experiments were done in triplicates.

### Example 3.I.f

### Soft Agar Colony Formation Assay

Plates with base agar were prepared 30 minutes before cell plating. The base agar was prepared by mixing equal volumes of 1% agarose at 40° C. and pre-warmed2×DMEM/10% FBS and poured onto a 6-well plate (1.5 mL per well) and allowed to polymerize.

After 30 minutes incubation, at room temperature equal volumes of warm 0.7% agarose and 2×DMEM/10% FBS (total volume 2 ml per well) were mixed with five thousands of transfected cells. The mix with cells was immediately poured on the base agar. The plates were incubated at 37° C. in a humidified incubator for 14-21 days. Colonies were stained with 0.5 mL of 0.005% Crystal Violet for >1 hour and counted using a dissecting microscope.

### Example 3.I.g

### Statistical Analysis

All assays were performed in triplicates and statistical evaluations were performed using the SPSS software package (bivariate correlations-Pearson coefficient and T-tests). All values in the text and figures represent the means±SD.

### Example 3.II

### Effects of hsa-miR29c* Mimic Delivery

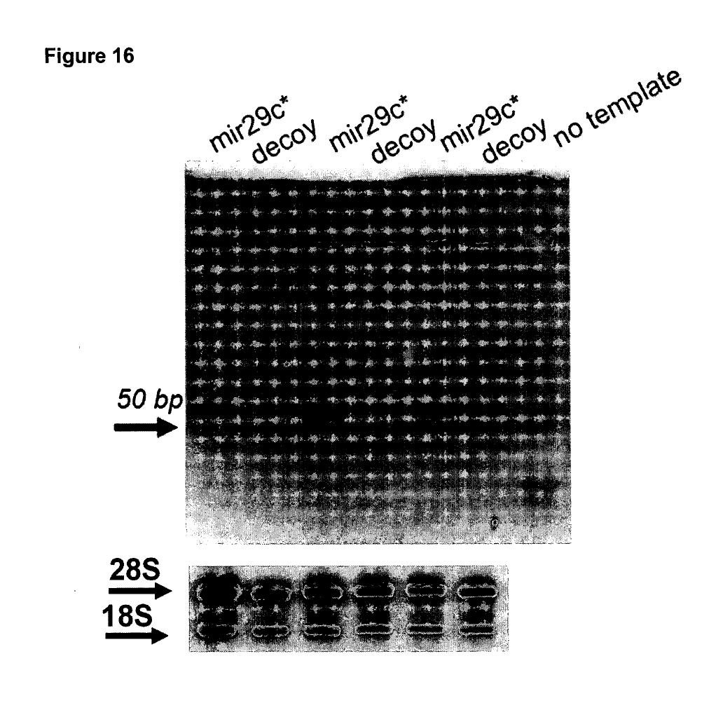

hsa-miR 29c* mimic was successfully transfected in all three cell lines studied, as seen in FIG. 16.

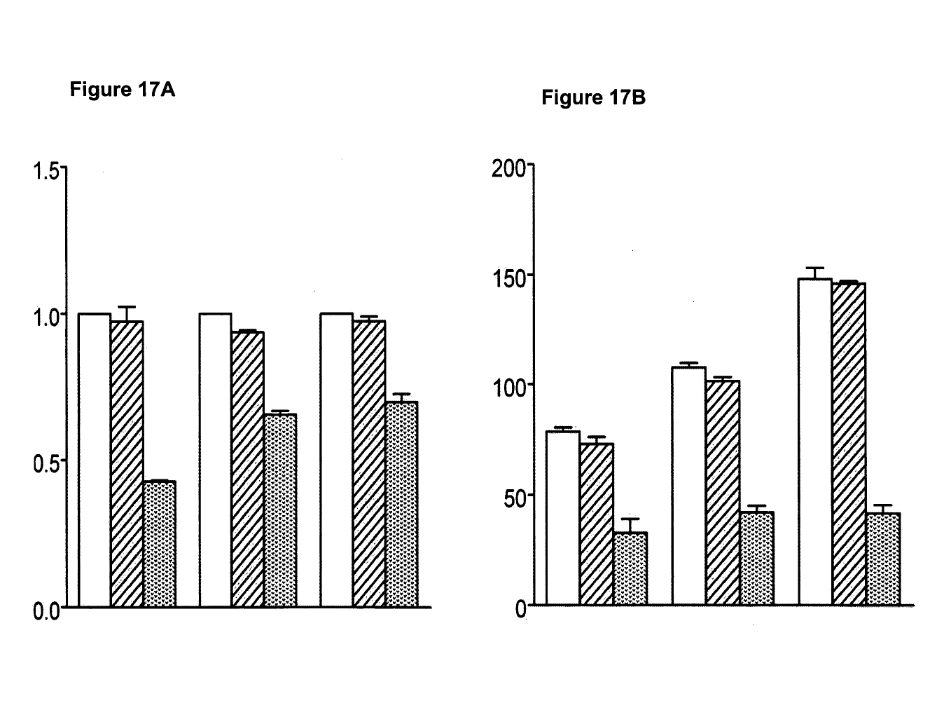

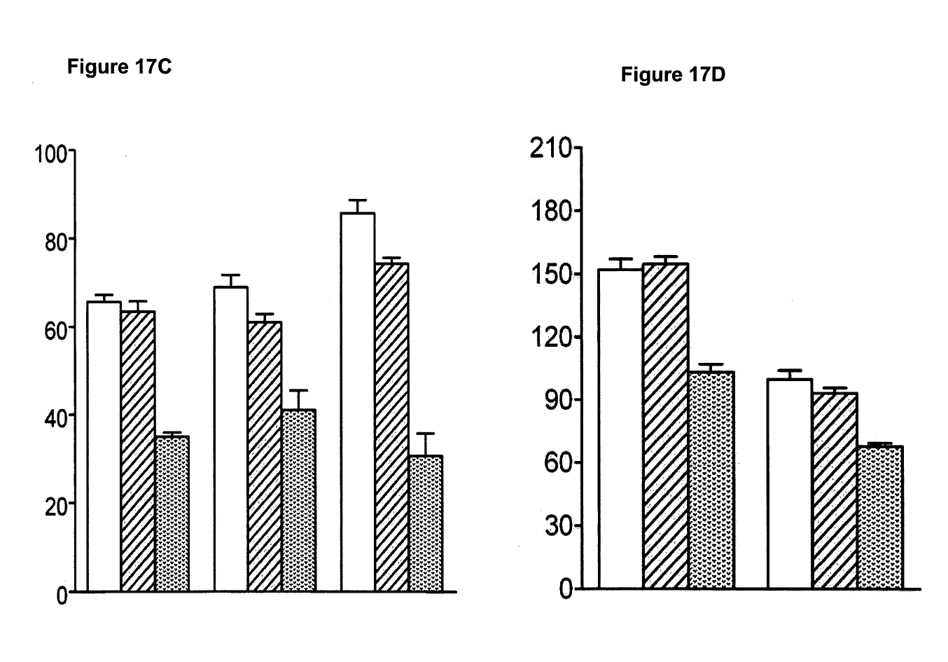

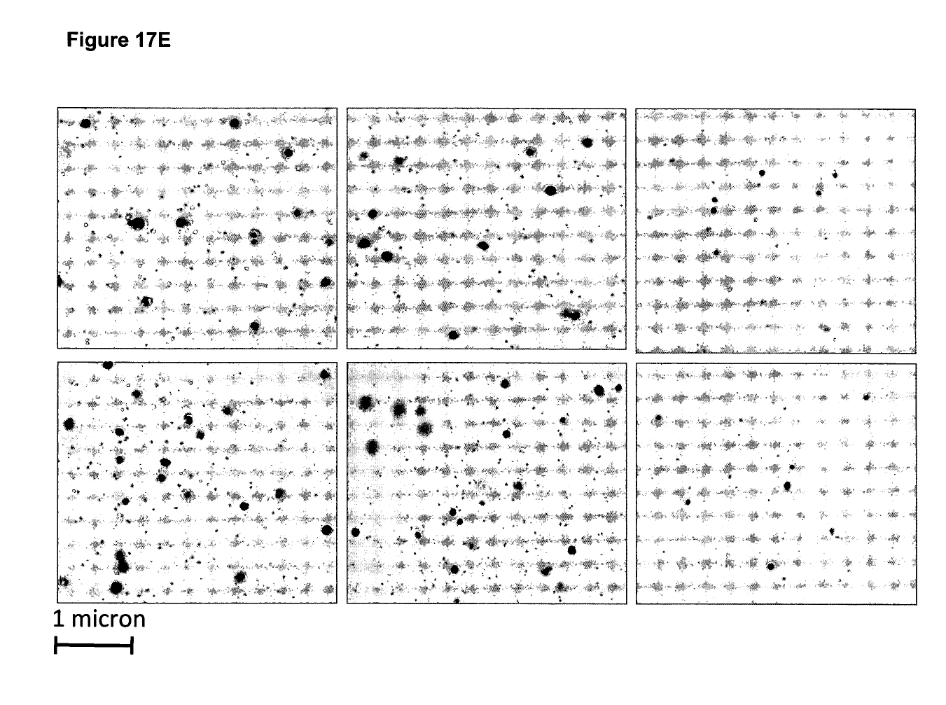

As indicated in FIGS. 17A-17C respectively, both lipofectin transfection, and transfection of a negative control had no impact on cell proliferation, invasion or migration. However, overexpression of hsa-miR 29c* (SEQ ID NO: 1) was associated with decreased proliferation, decreased migration, as well as a significant decrease in the invasive capacity of all three cell lines.

Additionally, as indicated in FIGS. 17D-17E, colony formation was inhibited with colonies being smaller and fewer with overexpression of hsa-miR 29c* (SEQ ID NO: 1).

### Example 4

### In Vivo Proof of Principle that Upregulation of miR29c* Decreases Tumor Growth and Invasion

A powerful lentiviral expression vectors with specific 29c* miRNA are used to enhance expression the miR in order to investigate its effect on the proliferation and metastasis of H-meso mesothelioma cells, which grow well in the flanks of nude mice. H-meso cells are ex-vivo infected with lentiviral particles and then transplanted into the flanks of the animals.

### Example 4.I

### Vector Construction of miR-29c*

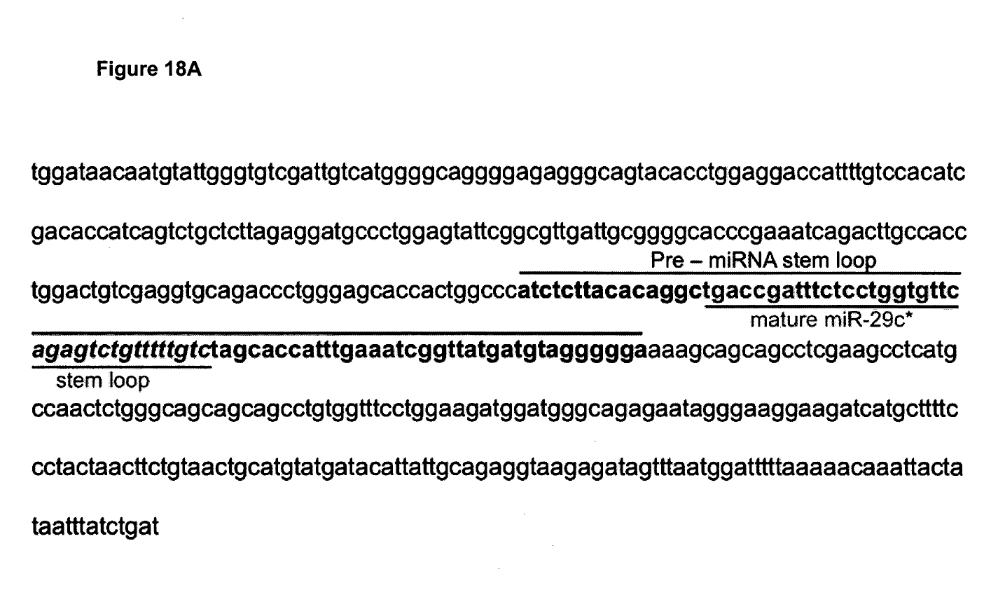

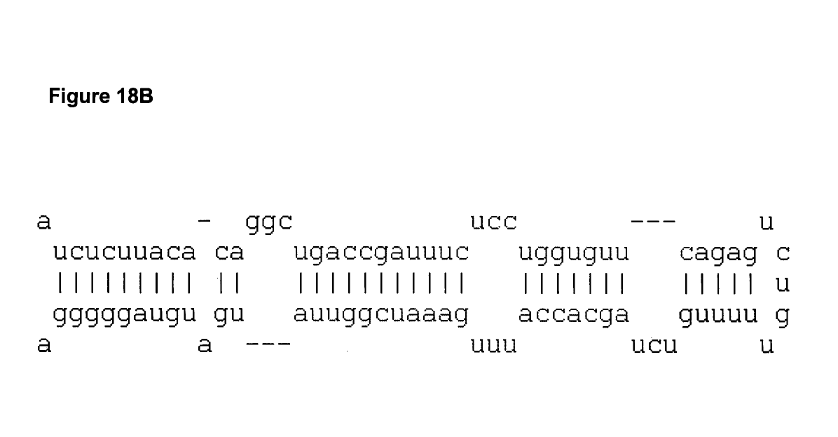

The miR-29c* expression construct consists of native stem loop and 200-400 base pairs of upstream and downstream flanking genomic sequence. The genomic DNA fragment containing the hsa-miR-29c* locus situated at chromosome 1 (1q32.2) plus 100-200 bp upstream and 100-200 bp downstream flanking genomic sequences from the 88 bp miR-29c* (FIG. 18) (SEQ ID NO:98) is amplified and cloned into a FIV-based pMIF-cGFP-Zeo-miR lenti-vector (System Biosciences). A scrambled negative control is also generated. A standard Lipofectamine protocol (Invitrogen) is used to generate Infectious lenti-viral particles using pPACKF1 lenti-viral packaging mix (System Biosciences) for both LENTI.29c* and LENTI.scrambled and viral titer is calculated. The produced infectious lenti-viral particle is introduced into the target cells.

### Example 4.II

### In Vivo Assays for H-Meso Tumor Growth and Metastasis

H-meso cells (5×10⁶) transfected with Lenti.29c* or Lenti.scrambled (at a MOI of 50) are implanted subcutaneously into the flank of nude mice (6 in each group, male BALB/c nu/nu, 4-6 weeks), and H-meso cells treated with phosphate-buffered saline are used as a mock control. Tumor growth is monitored with tumor volume, which is calculated as described: V (mm3)=width² (mm²)×length (mm)/2. The mice are sacrificed 6 weeks later, and the lungs are removed. Consecutive sections are made for every tissue block of the lung and stained with hematoxylin-eosin. The incidence and classification of lung metastasis are calculated and evaluated independently by two pathologists. Based on the number of H-meso cells in the maximal section of the metastatic lesion, the lung metastases are classified into four grades: grade I, <20 cells; grade II, 20-50 cells; grade III, 50-100 cells; and grade IV, >100 cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1. A method for determining a prognosis for mesothelioma in a subject, the method comprising:
(a) obtaining a biological sample from the subject;
(b) determining the expression level in said sample of a nucleic acid sequence selected from the group consisting of SEQ ID NOS: 1-14, 38-41, 50, 53-55, 6163 and sequences at least about 80% identical thereto; and
(c) comparing said expression level to a threshold expression level, wherein the comparison of the expression level of said nucleic acids to said threshold expression level is indicative of the prognosis of said subject.

2. The method of claim 1, wherein an expression level of a nucleic acid sequence selected from the group consisting of SEQ ID NOS: 5-6, 11-12 and sequences at least about 80% identical thereto, above said threshold expression level, is indicative of poor prognosis in said subject.

3. The method of claim 1, wherein an expression level of a nucleic acid sequence selected from the group consisting of SEQ ID NOS:1-2, 8, 50 and sequences at least about 80% identical thereto, below said threshold expression level, is indicative of poor prognosis in said subject.

4. The method of claim 1, wherein the prognosis is to predict overall survival of said subject.

5. The method of claim 1, wherein the prognosis is to predict the progression of mesothelioma in said subject.

6. A method for the diagnosis of mesothelioma in a subject, the method comprising:
(a) obtaining a biological sample from the subject;
(b) determining the expression level in said sample of a nucleic acid sequence selected from the group consisting of SEQ ID NOS: 11-49, 51-52 and sequences at least about 80% identical thereto; and
(c) comparing said expression level to a control expression level, wherein the comparison of the expression level of said nucleic acid compared to said control expression level is indicative of mesothelioma in said subject.

7. The method of claim 6, wherein an expression level of a nucleic acid sequence selected from the group consisting of SEQ ID NOS: 11-12, 34-35, 40-41, 46-47 and sequences at least about 80% identical thereto above said control expression level, is indicative of mesothelioma in said subject.

8. The method of claim 6, wherein an expression level of a nucleic acid sequence selected from the group consisting of SEQ ID NOS: 13-33, 36-39, 51-52 and sequences at least about 80% identical thereto, below said control expression level, is indicative of mesothelioma in said subject.

9. The method of claim 1, wherein the subject is a human.

10. The method of claim 1, wherein said method is used to determine a course of treatment for said subject.

11. The method of claim 1, wherein said biological sample is selected from the group consisting of bodily fluid, a cell line and a tissue sample.

12. The method of claim 11, wherein said tissue is a fresh frozen, fixed, wax-embedded or formalin fixed paraffin-embedded (FFPE) tissue.

13. The method of claim 12, wherein said tissue is mesothelium.

14. The method of claim 11, wherein said bodily fluid is serum.

15. The method of claim 1, wherein the expression levels are determined by a method selected from the group consisting of nucleic acid hybridization, nucleic acid amplification, and a combination thereof.

16. The method of claim 15, wherein the nucleic acid hybridization is performed using a solid-phase nucleic acid biochip array or in situ hybridization.

17. The method of claim 15, wherein the nucleic acid amplification method is real-time PCR.

18. The method of claim 17, wherein the real-time PCR method comprises forward and reverse primers.

19. The method of claim 18, wherein the forward primer comprises a sequence selected from the group consisting of SEQ ID NOS: 70-81 and sequences at least about 80% identical thereto.

20. The method of claim 18, wherein the real-time PCR method further comprises hybridization with a probe.

21. The method of claim 20, wherein the probe comprises a sequence selected from the group consisting of SEQ ID NOS: 82-93 and sequences at least about 80% identical thereto.

22. A kit for determining a diagnosis of mesothelioma, said kit comprises a probe comprising a nucleic acid sequence that is complementary to a sequence selected from the group consisting of SEQ ID NOS: 11-49, 51-52, a fragment thereof and a sequence at least about 80% identical thereto.

23. A kit for determining a prognosis of mesothelioma, said kit comprising a probe comprising a nucleic acid sequence that is complementary to a sequence selected from the group consisting of SEQ ID NOS: 1-14, 38-41, 50, 53-55, 61-63; a fragment thereof and a sequence at least about 80% identical thereto.

24. The kit of claim 22, wherein the kit further comprises forward and reverse primers.

25. The kit of claim 23, wherein said probe comprising a nucleic acid sequence selected from the group consisting of SEQ ID NOS: 82-88; a fragment thereof and a sequence at least about 80% identical thereto.

26. A method of treating or preventing mesothelioma in a subject in need thereof comprising administering to the subject an effective amount of a composition comprising a nucleic acid sequence selected from the group consisting of SEQ ID NOS: 1-2, 98, a fragment thereof and a sequence at least about 80% identical thereto.

27. (canceled)
